(19) United States
(12) Patent Application Publication
(10) Pub. No.: US 2014/0134261 A1
(43) Pub. Date: May 15, 2014

(54) Pharmaceutical Compositions Comprising Capsaicin Esters for Treating Pain and Cold Sores

(71) Applicants: Chandra U. Singh, San Antonio, TX (US); David L. Woody, New Braunfels, TX (US); Rao Nulu Jagaveerabhadra, Austin, TX (US)

(72) Inventors: Chandra U. Singh, San Antonio, TX (US); David L. Woody, New Braunfels, TX (US); Rao Nulu Jagaveerabhadra, Austin, TX (US)

(73) Assignee: Trinity Laboratories, Inc., San Antonio, TX (US)

(21) Appl. No.: 13/972,259

(22) Filed: Aug. 21, 2013

Related U.S. Application Data
(60) Provisional application No. 61/691,614, filed on Aug. 21, 2012.

(51) Int. Cl. A61K 31/573; A61K 31/05; A61K 31/618; A61K 31/045; A61K 31/192; A61K 31/10; A61K 31/197; A61K 31/135; A61K 31/215; A61K 35/56

(52) U.S. Cl. CPC A61K 31/573; A61K 31/215; A61K 31/05; A61K 31/618; A61K 35/57; A61K 31/192; A61K 31/10; A61K 31/197; A61K 31/135; A61K 31/045
USPC 424/522; 514/529; 514/162; 514/171; 514/552

(57) ABSTRACT

The present invention relates to pharmaceutical compositions comprising ester(s) of capsaicin and at least one other agent selected from salicylates, menthol, boswellic acids, DMSO, methyl sulfonylmethane, NSAIDs, corticosteroids, emu oil, opioid agonists and antagonists, NMDA antagonists, tramadol, hyaluronic acid, α2δ ligands, santalol, santalyl acetate, amyris alcohol, amyris acetate, aloe vera gel and aloe vera juice, for improved therapeutic properties. Further, the present invention relates to pharmaceutical compositions comprising high concentrations of ester(s) of capsaicin. Further, the present invention relates to a method of relieving pain due to various diseases in subjects by administering the pharmaceutical compositions of the invention. Further, the present invention relates to methods of relieving fever blisters due to cold sores in subjects by administering the pharmaceutical compositions comprising an ester of capsaicin and one other agent selected from santalol, santalyl acetate, amyris alcohol and amyris acetate.

### CROSS REFERENCE TO RELATED APPLICATIONS

This application claims the benefit of U.S. Appl. No. 61/691,614, filed Aug. 21, 2012. The content of the aforesaid application is relied upon and incorporated by reference in its entirety.

### FIELD OF THE INVENTION

The present invention generally relates at least to the fields of medicine and therapeutics, in particular to the fields of pain and cold sores.

### BACKGROUND OF THE INVENTION

Capsicum consists of the dried ripe fruits of Capsicum annuum Roxb. (Family Solanaceae), a small erect shrub indigenous to tropical America, cultivated in South America, China, India and Africa. Capsicum contains a crystalline pungent principle capsaicin, traces of a liquid alkaloid, red coloring matter and fatty oil. In folk medicine, capsicum is regarded as an aphrodisiac, depurative, digestive, stomachic, carminative, antispasmodic, diaphoretic, antiseptic, counterirritant, rubefacient, stypic, and tonic. Internally, capsicum has been used to treat asthma, pneumonia, diarrhea, cramps, colic, toothache, flatulent dyspepsia without inflammation; insufficiency of peripheral circulation; as a gargle for sore throat, chronic pharyngitis and laryngitis; and externally as a lotion or ointment to treat neuralgia, including rheumatic and arthritic pain, and unbroken chilblains (cold injuries) (Duke J. (1985). CRC Handbook of Medicinal Herbs. Boca Raton: CRC Press; Newall C A, et al., Herbal Medicines: A Guide for Health Care Professionals. London: Pharmaceutical Press, 1996).

The most potent and predominant chemical entity in capsicum is capsaicin (0.14%) (Cordell G A, Araujo O E., Capsaicin: identification, nomenclature, and pharmacotherapy. Annals of Pharmacotherapy. 1993: 27:330-336; FIG. 1). The heat sensation of pure capsaicin is approximately 16 million Scoville heat units (SHU) and is so hot that in its pure form diluted one hundred thousand fold it can cause blistering of the tongue. A series of homologous branched- and straight-chain alkyl vanillylamides, collectively known as capsaicinoids, is present in lesser concentrations than the parent compound, capsaicin. Of the capsaicinoid fraction, capsaicin (48.6%) is quantitatively followed by 6,7-dihydrocapsaicin (36%), nordihydrocapsaicin (7.4%), homodihydrocapsaicin (2%), and homocapsaicin (2%). Capsaicinoids and capsaicin are collectively found in amounts of 0.1% to 1%, with quantities varying according to soil and climate (Rumsfield, J A, and West D, Topical capsaicin in dermatological and peripheral pain disorders. DICP, Ann. Pharmacotherap. 1991; 25: 381-387).

Capsaicin has been studied since the mid-19th century and its structure is elucidated as 8-methyl-6-nonenoyl vanillylamide. Most pharmacological studies performed with isolated constituents of chile pepper have focused on capsaicin, which is the major pungent constituent. Nonivamide (pelargonic acid vanillylamide) is a common synthetic adulterant of capsicum products. Although structurally different from capsaicin, its presence in capsicum or capsaicin samples can be detected spectrographically and there is no evidence that this compound occurs naturally in Capsicum.

Capsaicin from edible chile peppers is allowed in human food by the U.S. FDA and other countries' health regulatory bodies. In the U.S., there is no maximum amount of capsaicin for food products. In the U.S., Capsicum spp. peppers are allowed as Generally Recognized as Safe (GRAS) under 21 CFR 182.10. Capsaicin extracted from plant sources are also GRAS under 21 CFR 182.20. Other regulations allow for the use of capsaicin in “Fever blister and cold sore treatment products” (21CFR310.545).

Capsaicin USP is a mixture of capsaicinoids (>95%), namely capsaicin (>60%), dihydrocapsaicin (>20%) and nordihydrocapsaicin (<15%). Capsaicin pure contains more than 97% capsaicin.

Capsaicin has played an important role in medicine for treating burning pain with a substance which causes burning pain (Szallasi A, et al., Vanilloid (capsaicin) receptors and mechanisms. Pharmacol Rev 1999; 51: 159-212). Creams, lotions, and patches containing capsaicin, generally in the range of 0.025-0.1% by weight, are now sold in many countries, often without the requirement of a prescription, for the management of neuropathic and musculoskeletal pain. Clinical studies of these medications, usually involving three to five topical skin applications per day for periods of 2-6 weeks, have generally suggested modest beneficial effects against various pain syndromes, including post-herpetic neuralgia (PHN), diabetic neuropathy, and chronic musculoskeletal pain (Derry S, et al., Topical capsaicin for chronic neuropathic pain in adults. Cochrane Database Syst Rev 2009; CD007393; Hempenstall K, et al., Analgesic therapy in postherpetic neuralgia: a quantitative systematic review. PLoS Med 2005; 2: e164). Since low-concentration, capsaicin-based products often result in contamination of the patient's environment (clothing, bedding, contact lenses, etc.) and each application may be associated with a burning sensation, poor patient compliance with these products is often cited as a likely contributor to limited efficacy (Altman R, Barkin R L. Topical therapy for osteoarthritis: clinical and pharmacologic perspectives. Postgrad Med 2009; 121: 139-47).

In an attempt to evaluate whether pain relief could be achieved by a single exposure to a much higher concentration of topical capsaicin, 10 patients with intractable pain syndromes were treated with a compounded high-concentration 5-10% w/w cream (Robbins W R, et al., Treatment of intractable pain with topical large-dose capsaicin: preliminary report. Anesth Analg 1998; 86: 579-83).

Patients were provided regional anaesthesia for tolerability and airborne contamination of treatment rooms occurred. Based on encouraging results, a high-concentration capsaicin-containing (8%) patch designated NGX-4010 and then given the trade name Qutenza™ was developed and evaluated (McCormack P L, Capsaicin dermal patch: in non-diabetic peripheral neuropathic pain. Drugs 2010; 70: 1831-42).

The capsaicin 8% patch is designed to rapidly deliver capsaicin into the skin while minimizing unwanted systemic or environmental exposure of capsaicin to patients and health-care providers. In 2009, Qutenza™ was approved for the treatment of peripheral neuropathic pain in non-diabetic adults in the EU, and in the USA to manage neuropathic pain associated with PHN. One important aspect of this formulation relative to low-concentration capsaicin formulations is removal of the potential for variability in administration and a lack of patient compliance, as its use occurs under the supervision of a health-care professional, and it requires a single application for 30 or 60 min. Furthermore, the environmental contamination issues associated with home use are avoided.

### Mechanism of Action of Capsaicin

A persistent confusion which continues to appear in the medical literature involves the role of ‘substance P depletion’ in capsaicin-induced pain relief. The neurogenic inflammation which follows application of topical capsaicin is due to the vascular actions of substance P and calcitonin gene-related peptide (CGRP) released from C-fibres. There is no evidence that the neurogenic inflammation which accompanies topical capsaicin administration is related to prolonged pain relief, even though it has long been appreciated that systemic capsaicin can cause substance P release by nociceptors (Jessell T M, et al., Capsaicin-induced depletion of substance P from primary sensory neurones. Brain Res 1978; 152: 183-8). In the early and mid-1980s, researchers observed that skin substance P levels were also significantly reduced after topical treatment with capsaicin (Bernstein J E, et al., Inhibition of axon reflex vasodilatation by topically applied capsaicin. J Invest Dermatol 1981; 76: 394-5). At that time, substance P was thought to be a fundamentally important signal for pain neurotransmission (hence the substantial efforts to develop substance P receptor antagonists), and the coincidental reduction of substance P content was inferred to play a causal relationship in capsaicin-induced pain relief. Since then, substance P receptor antagonists have failed as analgesics in a number of clinical trials (Hill R, NK1 (substance P) receptor antagonists—why are they not analgesic in humans? Trends Pharmacol Sci 2000; 21:244-6), and it is now widely recognized that of all the neuropeptides released by C-fibres, CGRP is a more likely potential contributor to pain pathophysiology, particularly in migraine (Fischer M J, Calcitonin gene-related peptide receptor antagonists for migraine. Expert Opin Investig Drugs 2010; 19: 815-23). If nociceptive nerve fibres retract from the epidermis and dermis then all markers they contain will be lost, and substance P is just one of many. The reduction of substance P content in skin after topical capsaicin administration is thus consequent to this process of nerve fibre defunctionalization and retraction. The ‘substance P depletion’ hypothesis was used to describe the mechanism of action of the low-concentration capsaicin formulations which became available in the 1980s, and, unfortunately over the years, this hypothesis continues to be repeated even in recent review articles and textbooks.

Capsaicin is a highly selective and potent (low nanomolar affinity) exogenous agonist for the TRPV1 receptor, a trans-membrane receptor ion channel complex which provides integrated responses to temperature, pH, and endogenous lipids (Alawi K and Keeble J, The paradoxical role of the transient receptor potential vanilloid 1 receptor in inflammation. Pharmacol Ther 2010; 125: 181-95). Responsiveness of TRPV1 receptors to these activators is also highly regulated by the phosphorylation state of the channel complex, the presence of ancillary proteins, and an ever-growing array of putative allosteric modulators (Cortright D N, Szallasi A, TRP channels and pain. Curr Pharm Des 2009; 15: 1736-49). When activated by a combination of heat, acidosis, or endogenous/exogenous agonists, TRPV1 may open transiently and initiate depolarization mediated by the influx of sodium and calcium ions. In the nociceptive sensory nerves which selectively express TRPV1 (mostly C- and some Aδ-fibers), depolarization results in action potentials, which propagate into the spinal cord and brain, and may be experienced as warming, burning, stinging, or itching sensations (FIG. 2; Anand P and Bley K. Topical capsaicin for pain management: therapeutic potential and mechanisms of action of the new high-concentration capsaicin 8% patch. British Journal of Anaesthesia 107 (4): 490-502 (2011)).

In contrast to transient activation which follows normal environmental stimuli or inflammatory responses to tissue injury, activation of TRPV1-expressing nerve fibers by exposure to a chemically stable exogenous agonist, such as capsaicin, can generate a biochemical signal with a persistent effect. The TRPV1 channel is highly calcium permeable, with calcium:sodium permeability ratio that starts at about 8:1 and increases to about 25:1 during prolonged capsaicin exposures, (Chung M K, et al., TRPV1 shows dynamic ionic selectivity during agonist stimulation. Nat Neurosci 2008; 11:555-64), which allows significant amounts of calcium to flow down its steep electrochemical gradient into nerve fibers. Furthermore, as TRPV1 is also expressed on intracellular organelles, external capsaicin application can cause release of calcium from the endoplasmic reticulum (Gallego et al., The endoplasmic reticulum of dorsal root ganglion neurons contains functional TRPV1 channels. J Biol Chem 2009; 284: 32591-601) and induce additional intracellular calcium release from internal stores via calcium-dependent calcium release (Huang W, et al., Transient receptor potential vanilloid subtype 1 channel mediated neuropeptide secretion and depressor effects: role of endoplasmic reticulum associated Ca2+ release receptors in rat dorsal root ganglion neurons. J Hypertens 2008; 26: 1966-75). Taken together, these multiple sources of calcium provide a robust intracellular signal which can overwhelm local calcium sequestration mechanisms. Consequently, sustained high levels of intracellular calcium can activate calcium-dependent enzymes such as proteases (Chard P S, et al., Capsaicin-induced neurotoxicity in cultured dorsal root ganglion neurons: involvement of calcium-activated proteases. Neuroscience 1995; 65:1099-108), and can induce the depolymerization of cytoskeletal components such as microtubules (Goswami C, et al., TRPV1 at nerve endings regulates growth cone morphology and movement through cytoskeleton reorganization. FEBS J 2007; 274: 760-72). In accord with these widely recognized effects, if TRPV1-expressing sensory nerve fibers are exposed to high concentrations of capsaicin or to lower concentrations in a continuous fashion, high levels of intracellular calcium and the associated enzymatic, cytoskeletal, and osmotic changes, and the disruption of mitochondrial respiration lead to impaired local nociceptor function for extended periods (FIG. 3; Bley K R. TRPV1 agonist approaches for pain management. In: Gomtsyan A, Faltynek C R, eds. Vanilloid Receptor TRPV1 in Drug Discovery: Targeting Pain and Other Pathological Disorders. New York: Wiley, 2010, 325-47; P. Anand and K. Bley. Topical capsaicin for pain management: therapeutic potential and mechanisms of action of the new high-concentration capsaicin 8% patch. British Journal of Anaesthesia 107 (4): 490-502 (2011)).

The term ‘desensitization’ is often used to describe these local effects of capsaicin on sensory nerve function, but is unsatisfactory in several respects. In the continued presence of exogenous agonists such as capsaicin, pharmacological desensitization of TRPV1 itself may indeed contribute acutely to analgesic efficacy. However, transient effects on TRPV1 are quite unlikely to account for the persistent pain relief seen clinically after either single treatments with high-concentration capsaicin or repetitive administration of low-concentration capsaicin. Hence, the emerging preferred term for the persistent local effects of capsaicin is ‘defunctionalization’ (Holzer P. The pharmacological challenge to tame the transient receptor potential vanilloid-1 (TRPV1) nocisensor. Br J Pharmacol 2008; 155: 1145-62), which avoids conceptual confusion with the intrinsic desensitisation of the TRPV1 receptor.

There is no evidence that topical capsaicin works through a transdermal systemic delivery into tissues other than the skin (FIG. 4; P. Anand and K. Bley, Topical capsaicin for pain management: therapeutic potential and mechanisms of action of the new high-concentration capsaicin 8% patch. British Journal of Anaesthesia 107 (4): 490-502 (2011)). Indeed, capsaicin is a very lipophilic, non-water-soluble compound and resists diffusion into aqueous solutions such as blood, and shows limited potential for transdermal delivery across human skin. Even when capsaicin is absorbed systemically, the duration of exposure is very short. The oral bioavailability of capsaicin was recently reported in humans: after ingestion of 26.6 mg of capsaicin, the pharmacokinetic parameters were a Cₘₐₓ of 2.5 (0.1) ng ml⁻¹, Tₘₐₓ of 47.1 (2.0) min, and T_{1/2} of 24.9 (5.0) min (Chaiyasit K, et al., Pharmacokinetics and the effect of capsaicin in Capsicum frutescens on decreasing plasma glucose level. J Med Assoc Thai 2009; 92:108-13). There are no published data from low-concentration formulations, but after 60 or 90 min capsaicin 8% patch treatments for painful peripheral neuropathy, plasma concentrations were also very low (with a population Cₘₐₓ of 1.86 ng ml⁻¹) and transient (mean elimination half-life of 1.64 h) (Babbar S, et al., Pharmacokinetic analysis of capsaicin after topical administration of a high-concentration capsaicin patch to patients with peripheral neuropathic pain. Ther Drug Monit 2009; 31: 502-10). The longer elimination half-life of topical capsaicin relative to oral exposure is likely to reflect its slow release from the skin at the patch application site. Capsaicin is metabolized rapidly by several cytochrome (CYP) enzymes present in the human liver, but in vitro studies show that its metabolism in human skin is quite slow (Chanda S, et al., In vitro hepatic and skin metabolism of capsaicin. Drug Metab Dispos 2008; 36: 670-5). The implication for topical capsaicin-containing analgesics is that capsaicin can reside at the site of action (i.e. skin) relatively unchanged, whereas any capsaicin which is transdermally absorbed is rapidly eliminated.

### Other Benefits of Capsaicin

Apart from its pain relieving property, capsaicin has several other beneficial effects in humans which have been described below.

### a) Energy Metabolism

It has been shown that capsaicin added to meals increased sympathetic nervous system activity and energy metabolism (Yoshioka M, et al., Effects of red pepper on appetite and energy intake, Br. J. Nutr. 1999; 82: 115-123; Yoshioka M., et al, Effects of red pepper added to high-fat and high-carbohydrate meals on energy metabolism and substrate utilization in Japanese women, Br. J. Nutr. 1999; 80: 503-510; Lejeune M P., et al., 2003, Effect of capsaicin on substrate oxidation and weight maintenance after modest body-weight loss in human subjects, Br. J. Nutr. 90: 651-659).

Capsaicin affects lipid metabolism as demonstrated in a study by Kawada et al. (Effects of capsaicin on lipid metabolism in rates fed a high fat diet; Journal of Nutrition, 1986; 116, 1272-78). Male rats fed a diet containing 30% lard with capsaicin at 0.14% of the diet developed serum triglyceride levels that were significantly lower than those of animals receiving a high-fat diet without capsaicin. But levels of free fatty acids, cholesterol, and pre-beta-lipoprotein were not affected. Activities of liver enzymes involved in lipid synthesis (acetyl-CoA carboxylase) and in carbohydrate metabolism (glucose-6-phosphate dehydrogenase) were inhibited in the high-fat diet, but the activity of the latter was restored to control levels by the added dietary capsaicin. The weight of perirenal adipose tissue was reduced in a dose-dependent manner by capsaicin. These results suggested that capsaicin did not interfere with lipid biosynthesis. Rather, that capsaicin might stimulate lipid metabolism, and possibly facilitates mobilization of lipid from adipose tissue.

In a follow-up to the study above, Kawada et al. (Capsaicin-induced beta-adrenergic action on energy metabolism in rats: influence of capsaicin on oxygen consumption, the respiratory quotient, and substrate utilization, Proc Soc Exp Biol Med. 1986; 183(2):250-6) measured the effect of i.p. administered capsaicin on general energy metabolism, including oxygen consumption, respiratory quotient, and substrate utilization. Capsaicin had a general stimulatory effect on metabolism, similar to that of epinephrine; oxygen consumption was elevated, respiratory quotient was initially elevated, then decreased; and serum glucose and insulin levels were elevated, concomitant with a rapid decrease in liver glycogen, and a gradual increase in serum triglycerides. The response was blocked by beta-adrenergic blockers, but was not affected by alpha-adrenergic or ganglion blockers. Their results suggested that capsaicin effects metabolism either as a direct beta-adrenergic agonist, or indirectly by stimulating catecholamine release.

### b) Cardiovascular Effect

Yamato et al. (Inhibition of contractile tension by capsaicin in isolated rat papillary muscle. Gen. Pharmac, 1996; 27: 129-132) showed that capsaicin produced a marked concentration-dependent decrease in the amplitude, the rate of rise, and the rate of relaxation of the contractile tension of rat ventricular papillary muscles; however, the half-life of the relaxation and the time to peak tension were only slightly effected. Calcium release and shortening of action potential duration in ventricular myocytes was profoundly reduced by capsaicin, perhaps resulting from the non-specific membrane-stabilizing effects of capsaicin.

Capsaicin treatment caused a biphasic effect on contractile force, left ventricular systolic blood pressure, and heart rate of isolated perfused rat hearts. A transient initial increase in contractile force and left ventricular systolic pressure was observed, followed by a prolonged depression of both parameters. Heart rate was increased, but this effect was not followed by a subsequent reduction.

The initial increases in contractile force and blood pressure could have been induced by the release of calcitonin-gene-related peptide (CGRP) from local sensory nerves; the negative inotropic effects following the initial increase may be due to a direct inhibitory effect of capsaicin on ventricular cells, or to nonspecific membrane-stabilizing effects. The increased heart rate was attributed to the release of CGRP (Lundberg J M, et al., Co-existence of substance P and calcitonin gene-related peptide-like immunoreactivities in sensory nerves in relation to cardiovascular and bronchoconstrictor effects of capsaicin. Eur. J. Pharmacol., 1985; 108, 315-319).

Capsaicin elicits a vasoconstrictive response in the large cerebral arteries of the cat (Saito A & Goto K, Depletion of calcitonin gene-related peptide (CGRP) by capsaicin in cerebral arteries. J. Pharmaco biodyn., 1986; 9, 613-619), and in the middle and basilar cerebral arteries, an effect was attributed to a direct contraction of smooth muscle, since the response was independent of the presence of endothelium and nerve components. It acts on the vanilloid (TRPV1) receptors of perivascular sensory nerve fibres and releases their neuropeptide content, resulting in vasodilatation, while capsaicin-induced vasoconstriction is probably a direct effect on blood vessels by calcium inflow into the smooth muscle cells (Edvinsson L, et al., Cerebrovascular responses to capsaicin in vitro and in situ. Br J Pharmacol, 1990; 100:312-318).

### c) Effect on Migraine

An increased activity of CGRP-containing trigeminovascular nerve fibres has been correlated to the pathophysiology of migraine (Buzzi M G, et al., Dihydroergotamine and sumatriptan attenuate levels of CGRP in plasma in rat superior sagittal sinus during electrical stimulation of the trigeminal ganglion. Neuropharmacology, 1991; 30, 1193-1200) either during attacks or as a general imbalance in migraine patients (Ashina M, et al., Evidence for increased plasma levels of calcitonin gene-related peptide in migraine outside of attacks. Pain, 2000; 86, 133-138). Capsaicin potently and selectively causes release of CGRP from sensory nerve terminals both in vitro and in vivo. The mechanism of capsaicin-induced CGRP depletion involves binding of capsaicin to vanilloid 1 receptors (VR1). Capsaicin-association to VRs triggers Ca2+ influx and elevated intracellular calcium levels in turn stimulates CGRP-release. Capsaicin is thought to activate the sympathetic nerves via vanilloid receptor 1 (VR-1) by stimulating the release of NE into the synaptic cleft (Vogel G, Hot pepper receptor could help manage pain, Science. 2000; 288: 241-242).

### d) Digestive and Gastrointestinal Effect

In tests using cultured human intestinal epithelial cells, Jensen-Jarolim et al. (Hot spices influence permeability of human intestinal epithelial monolayers. J Nutr. 1998; 128:577-81) found sufficient in vitro evidence to suggest that Capsaicin may increase the permeability of the gastrointestinal tract to allow transport of macromolecules and ions across the epithelium; an effect, they add, that might have importance to food intolerance and allergic reactions to food. The stimulatory effect of orally administered capsaicin on gastric acid secretion and mucosal blood flow was studied in rats using amounts roughly equivalent to a normal Thai diet. Capsaicin was noted to have a protective effect on gastric mucosa of ethanol-induced gastric lesions in rats (Uchida M, et al., The role of capsaicin-sensitive afferent nerves in protective effect of capsaicin against absolute ethanol-induced gastric lesions in rats. Jpn J Pharmacol, 1991; 55: 279-282). The protective effect was attenuated upon pretreatment with indomethacin and disappeared in capsaicin-sensitive nerve-degenerated rats, suggesting that enhanced prostaglandin formation inhibited lesion formation. Further study by the same group found decreased stomach motility and increased mucosal blood flow with intragastric capsaicin treatment, whereas capsaicin pre-treatment desensitized the afferent neurons, thereby mitigating this protective effect.

### e) Anti-Cancer Effect

An in vitro chemopreventive activity of capsaicin was shown by Morre et al. (Capsaicin inhibits preferentially the NADH oxidase and growth of transformed cells in culture. Proc. Natl. Acad. Sci. USA 1995; 92:183). When capsaicin was added to cultured cells of Caov-3 human ovarian carcinoma, MCF-10A human mammary adenocarcinoma, HL-60 human promyelocytic leukemia, and HeLa cells, a preferential growth-inhibition was evident as cells became smaller and underwent cell death. Condensed and appearing fragmented, the nuclear DNA of these cells suggested that capsaicin had induced apoptosis.

Capsaicin has a profound antiproliferative effect on prostate cancer cells (Mori A, et al., Capsaicin, a Component of Red Peppers, Inhibits the Growth of Androgen-Independent, p53 Mutant Prostate Cancer Cells. Cancer Res., 2006; 66(6): 3222-3229), inducing the apoptosis of both androgen receptor (AR)-positive (LNCaP) and -negative (PC-3, DU-145) prostate cancer cell lines associated with an increase of p53, p21, and Bax. Capsaicin down-regulated the expression of not only prostate-specific antigen (PSA) but also AR. Promoter assays showed that capsaicin inhibited the ability of dihydrotestosterone to activate the PSA promoter/enhancer even in the presence of exogenous AR in LNCaP cells, suggesting that capsaicin inhibited the transcription of PSA not only via downregulation of expression of AR, but also by a direct inhibitory effect on PSA transcription. Capsaicin inhibited NF-K activation by preventing its nuclear migration. In further studies, capsaicin inhibited tumor necrosis factor-A-stimulated degradation of IKBA in PC-3 cells, which was associated with the inhibition of proteasome activity. Taken together, capsaicin inhibits proteasome activity which suppressed the degradation of IKBA, preventing the activation of NF-KB. Capsaicin, when given orally, significantly slowed the growth of PC-3 prostate cancer xenografts as measured by size. These data suggests that capsaicin, or a related analogue, may have a role in the management of prostate cancer (Aggarwal B B, et al., Potential of spice-derived phytochemicals for cancer prevention. Planta Med 2008; 74:1560-9).

### f) Effect on Immune System

In vitro studies show that capsaicin exhibits anti-inflammatory properties. The prevention of release of pro-inflammatory mediators, eicosanoids, and hydrolytic enzymes is associated with the anti-inflammatory properties of capsaicin. Rat peritoneal macrophages pre-incubated with 10 μM capsaicin for 1 h inhibited the incorporation of arachidonic acid into membrane lipids, prostaglandin E2, leukotriene B4, and leukotriene C4 by 76%, 48%, 46%, and 48%, respectively (Joe B, Lokesh B R. Effect of curcumin and capsaicin on arachidonic acid metabolism and lysosomal enzyme secretion by rat peritoneal macrophages. Lipids 1997; 32: 1173-80). It has been shown that small-dose capsaicin pretreatment caused a significant decrease in the production of pro-inflammatory cytokines (TNF, IL-6) and was also associated with a marked increase in the production of the anti-inflammatory cytokine IL-10 in the rat model of sepsis, suggesting that in vivo pretreatment with small-dose capsaicin exerts a wide range of anti-inflammatory properties of capsaicin (Demirbilek S, et al., (2004) Small-dose capsaicin reduces systemic inflammatory responses in septic rats. Anesth Analg 99: 1501-1507). Such a decrease in pro-inflammatory cytokine levels could also explain, at least in part, why small-dose capsaicin treatment resulted in the ability of these animals to produce more of the IL-10 in response to CLP-induced sepsis.

Oxidative damage is probably one of several factors that lead to cell damage, organ dysfunction, and death. There is convincing evidence of severe oxidative stress in patients with sepsis (Macdonald J, et al., Oxidative stress and gene expression in sepsis. Br J Anaesth 2003; 90: 221-32.). Reactive oxygen species (ROS) and RNS, such as superoxide anions, peroxides, hydroxyl radicals, and nitric oxide (NO) generated by activated macrophages for defense mechanisms of the host, can also act as mediators of inflammation if produced in an uncontrolled manner. These radicals can react with cellular components like lipids, proteins, and nucleic acids, resulting in increased levels of lipid peroxides and alterations in the functions of proteins, and may also cause DNA damage. In vitro studies show that capsaicin has a potent antioxidant effect. Capsaicin inhibits generation of ROS (Joe B, Lokesh B R., Role of capsaicin, curcumin and dietary n-3 fatty acids in lowering the generation of reactive oxygen species in rat peritoneal macrophages. Biochim Biophys Acta 1994; 1224: 255-63). Preincubation of macrophages with 10 μM capsaicin completely inhibited the superoxide anions, hydrogen peroxide, and nitrite radical production in vitro by macrophages. In addition, it was reported that capsaicin potentially inhibits various lipid peroxidations. Capsaicin was found to scavenge radicals at and near the membrane surface and in the interior of the membrane (Kogure K, et al. Mechanism of potent antiperoxidative effect of capsaicin. Biochim Biophys Acta 2002; 1573: 84-92). Systemic administration of capsaicin at 1 mg/kg before cecal ligation and puncture decreased the lipid peroxidation in various tissues, including lung and liver, during the late sepsis period (Demirbilek S, et al., (2004) Small-dose capsaicin reduces systemic inflammatory responses in septic rats. Anesth Analg 99: 1501-1507). SOD levels were different between the septic rats pretreated with small-dose capsaicin and those that did not receive pretreatment. Oxidative stress was reduced in the septic rats pretreated with small-dose capsaicin.

Viral replication, immune regulation, and induction of various inflammatory and growth-regulatory genes require activation of a nuclear transcription factor (NF)-κ-B. Agents that can block NF-κ-B activation have potential to block downstream responses mediated through this transcription factor. Capsaicin (8-methyl-N-vanillyl-6-nonenamide) has been shown to regulate a wide variety of activities that require NF-κ-B activation (Singh S, et al., Capsaicin (8-methyl-N-vanillyl-6-nonenamide) is a potent inhibitor of nuclear transcription factor-κB activation by diverse agents. J Immunol 1996; 157:4412-20). The pretreatment of human myeloid ML-1a cells with capsaicin blocked TNF-mediated activation of NF-κ-B in a dose- and time-dependent manner. Capsaicin treatment of cells also blocked the degradation of I-κ-B alpha, and thus the nuclear translocation of the p65 subunit of NF-κ-B, which is essential for NF-κ-B activation. TNF-dependent promoter activity of I-κ-B alpha, which contains NF-κ⁻B binding sites, was also inhibited by capsaicin.

### q) Effect on HDL and LDL Cholesterol

The effects of capsaicin and dihydrocapsaicin on blood lipid and lipoprotein concentrations were determined in two groups of turkeys (Negulesco J A, et al, Effects of pure capsaicinoids (capsaicin and dihydrocapsaicin) on plasma lipid and lipoprotein concentrations of turkey poults. Atherosclerosis. 1987; 64(2-3):85-90). The first group was maintained on a cholesterol-free diet, while the second received a diet supplemented with 0.2% cholesterol. Daily administration of capsaicinoids occurred at a dose of 4 mg per animal. Neither drug had an effect on serum triglyceride concentrations in the animals receiving the cholesterol-free diet. However, total cholesterol, LDL-cholesterol and HDL-cholesterol concentrations were increased significantly, while VLDL cholesterol concentrations were decreased significantly by both drugs relative to controls. In the cholesterol-fed group triglycerides, total cholesterol and LDL-cholesterol decreased significantly with dihydrocapsaicin treatment. Both compounds reduced VLDL-cholesterol and increased HDL-cholesterol in the cholesterol-fed animals. Dihydrocapsaicin had a greater efficacy in producing beneficial anti-hyperlipidemic effects in the cholesterol-fed animals.

### h) Effect on Diabetes

Capsicum frutescens has been used to treat diabetes mellitus by traditional healers in Jamaica (Tolan I, et al., Isolation and purification of the hypoglycaemic principle present in Capsicum frutescens. Phytother Res 2004; 18:95-6). Purified capsaicin caused a decrease in blood glucose levels to 4.91+/−0.52 (n=6) mmol/dL versus 6.40+/−0.13 mmol/dL (n=6) for the control (p<0.05) at 2.5 h in an OGTT in dogs. There was a concomitant elevation in plasma insulin levels (p<0.05). Capsaicin is responsible for the hypoglycaemic episodes seen in the dogs. It is also apparent that the latter is mediated by insulin release.

U.S. Patent Application Pub. No. 2002/0058048 relates to a topical capsaicin preparation for the treatment of painful cutaneous disorders and neural dysfunction is disclosed. The preparation contains a nonionic, amphoteric or cationic surfactant in an amount effective to eliminate or substantially ameliorate burning pain caused by capsaicin.

U.S. Pat. No. 6,239,180 relates to transdermal application of capsaicin (or a capsaicin analog) in a concentration from greater than about 5% to about 10% by weight for treating neuropathic pain, so long as an anesthetic, preferably by means of a transdermal patch, is administered initially to the affected area to minimize the expected side effects from subsequent capsaicin application. Various analogs of capsaicin with physiological properties similar to capsaicin are known (Ton 1955). For example, resiniferatoxin is described as a capsaicin analog by Blumberg, U.S. Pat. No. 5,290,816. U.S. Pat. No. 4,812,446 also relates to capsaicin analogs and methods for their preparation.

U.S. Pat. No. 6,348,501 relates to a lotion for treating the symptoms of arthritis using capsaicin and an analgesic, and a method for making such lotion. U.S. Pat. No. 6,573,302 relates to a cream comprising: a topical carrier wherein the topical carrier comprises a member selected from the group comprising lavender oil, myristal myristate, and other preservatives including, hypericum perforatum arnica montana capric acid; and 0.01 to 1.0 wt. capsaicin; 2 to 10 wt. % of an encapsulation agent selected from the group comprising colloidal oatmeal hydrogenated lecithin, dipotassium glycyrlhizinate and combinations thereof; esters of amino acid; a light scattering element having a particle size up to 100 nm; and a histidine.

In 2009, the U.S. Food and Drug Administration (FDA) approved Qutenza® (capsaicin) 8% patch for the management of neuropathic pain due to postherpetic neuralgia (PHN), the nerve pain which can follow shingles. Qutenza® delivers through a dermal delivery system, providing up to 12 weeks of reduced pain following a single one-hour application. A numbing gel or cream has to be applied to the painful area and left on long enough to reduce discomfort associated with the Qutenza® patch application. The gel or cream is removed prior to applying Qutenza®. In clinical trials, the most common adverse reactions were application site redness, pain, itching, and papules. Among patients treated with Qutenza®, one percent discontinued prematurely due to an adverse event. Serious adverse reactions included application site pain and increased blood pressure. Increases in blood pressure occurred during or shortly after exposure to Qutenza®. The changes were on average less than 10 mm Hg, although some patients had greater increases and these changes lasted for approximately two hours after patch removal. The most common side effects of Qutenza® include redness, pain, small bumps, and itching, which occur at the treatment site right after Qutenza® is placed on the skin. In a 12-week study, the Qutenza group demonstrated a greater reduction in pain compared to the Control group during the primary assessment at week 8. The percent change in average pain from baseline to week 8 was −18% or the low-dose control and −29% for Qutenza®.

Other adverse reactions observed during the clinical studies of Qutenza® include application site urticaria, application site paresthesia, application site dermatitis, application site hyperesthesia, application site excoriation, application site warmth, application site anesthesia, application site bruising, application site inflammation, application site exfoliation, peripheral edema; Nervous System Disorders: headache, burning sensation, peripheral sensory neuropathy, dizziness, dysgeusia, hyperesthesia, hypoesthesia respiratory; Thoracic and Mediastinal Disorders: cough, throat irritation; and Skin and Subcutaneous Tissue Disorders: abnormal skin odor.

U.S. Pat. No. 4,599,342 (LaHann) relates to the combinations of capsaicin derivatives of the general formula

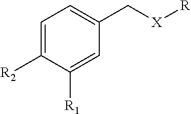

wherein R₁ is OH or OCH₃, R₂ is OH or a short-chain ester, ester, X is

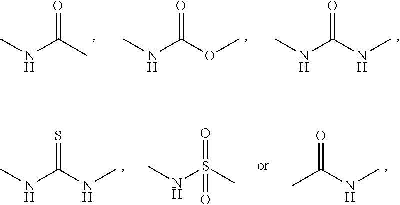

R is a C₅-C₁₁ alkyl, C₅-C₁₁ alkenyl, C₁₁-C₂₃ cis alkenyl, C₁₁-C₂₃ alkynyl, C₁₁-C₂₃ alkadienyl, or C₁₁-C₂₃ methylene substituted alkane, with an opioid analgesic, providing analgesic activity in humans and lower animals.

U.S. Pat. No. 7,244,767 (Bisogno et al.) relates to ester derivatives of capsaicin and the method for synthesis of such derivatives (page 13; line 5). The following capsaicin derivatives are recited:

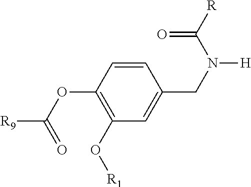

in which: a) R₁ is chosen from the group comprising hydrogen, linear or branched, saturated or unsaturated C1-C10 alkyl, C3-C7 cycloalkyl or C7-C10 arylalkyl; b) R₉ is a saturated or monounsaturated, linear or branched C1-C10 alkyl radical, or a cycloalkyl, arylalkyl or heterocyclic radical optionally substituted with one or more —OH, —COOH, —SO₃H, —NH₂, —NHR₆, —NR₄R₅, ⁺NR₄R₆R₆ Z⁻ groups; and c) R is: carboxyl, —COOR₇, saturated or unsaturated cycloalkyl, polycyclic alkyl, aryl, heteroaryl, arylalkyl or C1-C35 alkyl, which is saturated or unsaturated with 1 to 6 double bonds, linear or branched and unsubstituted or substituted. U.S. Pat. No. 7,632,519 (Jamieson et al.) relates to a compound of the formula:

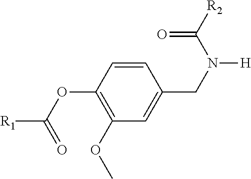

wherein R₁ is selected from the group consisting of hydrogen, —(CH₂)ₙCH₃ wherein n is an integer from 6-19, and a substituted, saturated or unsaturated, linear or branched, C₁-C₂₀ alkyl and R₂ is selected from the group consisting of a substituted or unsubstituted, unsaturated, linear or branched C₂-C₅ alkyl, or (3E)-2-methyloct-3-ene, or (3Z)-2-methyloct-3-ene.

U.S. Pat. No. 4,812,446 (Brand) relates to an analgesic composition comprising capsaicin or a capsaicin analogue and an analgesic selected from the class of non-steroidal anti-inflammatory, antipyretic and analgesic drugs.

U.S. Pat. No. 7,943,666 relates to formulations of ester derivatives of capsaicin and ester derivatives of myristoleic acid. Based on this invention, a commercial product called Paloxin® containing capsaicin palmitate at a concentration of 0.45% has been produced and marketed as a topical treatment for pain.

U.S. Pat. No. 7,645,767 relates to pharmaceutical compositions for treating chronic pain in a mammal suffering therefrom by administering to the mammal a chronic pain alleviating amount of a nontoxic N-methyl-D-aspartate receptor antagonist such as dextromethorphan, dextrorphan, ketamine or pharmaceutically acceptable salt thereof, in combination with a μ-opiate analgesic such as tramadol or an analogously acting molecular entity, and a capsaicin or an ester of capsaicin, and optionally in sustained release dosage form.

U.S. Patent Application Publication No. 2010/0120912 relates to nutraceutical or dietary supplemental compositions comprising esterified capsaicinoids. The esterified capsaicinoids would be converted to the active parent capsaicinoid compound following enzymatic or chemical hydrolysis. In various embodiments, these esterified capsaicinoids have a higher lipophilicity, lipid solubility and result in less irritation to the stomach than the parent capsaicinoid, and hence may be included in certain dietary supplement formulations, including capsules, pills and tablets. The dietary supplement compositions may be used for pain management in mammals in vivo and/or in the treatment of various pathological conditions in humans.

The art has yet to produce a capsaicin based topical treatment for treating pain containing more than 5% of capsaicin without the unwanted adverse effects including intense stinging pain at the site of application. The present inventors have unexpectedly discovered that topical ointment containing high concentrations of a capsaicin ester, such as capsaicin palmitate (e.g., about 14.25%; corresponding to about 8% of capsaicin) has almost no burning pain at the site of the application and does not rely on topical anesthetics, such as lidocaine (Entry 5310, p. 786 Merck Index, Tenth Edition (1983)) and benzocaine (ethyl aminobenzoate, Entry 3710, p. 546 Merck Index, Tenth Edition, (1983)), before the application of the ointment. On the other hand, the application of 8% capsaicin produced intense pain at the site of application within 15 minutes and the pain and inflammation lasted for almost a day.

Further, the inventors have discovered in an unexpected manner that the ester of capsaicin can be incorporated into pharmaceutical compositions containing other pain relieving agents such as salicylates, menthol, boswellic acids, DMSO, methyl sulfonylmethane, NSAIDs, corticosteroids, emu oil, opioid agonists and antagonists, NMDA antagonists, tramadol, hyaluronic acid, α2δ ligands, aloe vera gel and aloe vera juice.

### SUMMARY OF THE INVENTION

The present invention provides novel pharmaceutical compositions comprising ester derivatives of capsaicin that are highly lipophilic. Without being bound by theory, it is believed that the esters of capsaicin set forth herein are enzymatically cleaved to the parent compound, capsaicin. Thus, the compositions set forth herein provide for a novel form of therapy of diseases amenable to treatment with capsaicin.

The compositions comprising ester derivatives of capsaicin of the present invention will have significant advantage over compositions comprising capsaicin and existing derivatives currently described in the patent and scientific literature. In particular, in view of their high lipophilicity, non-irritation to the skin, almost non-burning sensation at the site of application and stability, these compositions are highly desirable for topical administration in high concentration as compared to capsaicin. In addition, because of their stability and non-toxic nature, these compositions can be made more readily available to the general public.

The inventors have surprisingly and unexpectedly discovered that compositions comprising high concentrations of ester derivatives of capsaicin have therapeutic utility in treating pain in subjects, without significant undesirable side effects. These compositions thus provide for a novel form of therapy of any disease or condition wherein capsaicin is believed to be of benefit, including but not limited to, post-herpetic neuralgia, shingles (herpes zoster), cold sores, diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

Further, the inventors have discovered in an unexpected manner that the ester(s) of capsaicin can be incorporated into pharmaceutical compositions containing other pain relieving agents such as salicylates, menthol, boswellic acids, DMSO, methyl sulfonylmethane, NSAIDs, corticosteroids, emu oil, opioid agonists and antagonists, NMDA antagonists, tramadol, hyaluronic acid, α2δ ligands, aloe vera gel and aloe vera juice for improved pain relieving properties.

Further, the ester(s) of capsaicin can be combined with santalol, santalyl acetate, amyris alcohol or amyris acetate for the treatment of cold sores and herpes.

The present invention generally pertains to pharmaceutical compositions containing a compound of formula (I):

R—CO-CAP (I)

wherein CAP refers to the capsaicin group represented in FIG. 5.

In formula I, R is selected from alkyl groups of ranging from 11 up to about 22 carbon atoms and aryl groups of ranging from 11 up to about 22 carbon atoms and alkylene group of ranging from 11 up to about 22 carbon atoms and an arylene group of ranging from 11 up to about 22 carbon atoms. The alkyl, aryl and alkylene groups may be substituted or unsubstituted, branched or straight chains. In addition, R may contain heteroatoms and may be straight chained or branched.

Examples of suitable straight-chain alkyl groups in formula I include but not limited to 1-hendecyl, 1-pentadecyl, 1-heptadecyl, 1-hexadecyl, 1-octadecyl and the like groups.

Among the compounds represented by the general Formula I, in some embodiments, R is one of the following groups: 1-hendecyl, 1-pentadecyl, 1-heptadecyl, 1-hexadecyl and 1-octadecyl.

The compounds of Formula I are esters of capsaicin which can be incorporated into a topical formulation in high concentration for treating diseases such as post-herpetic neuralgia, shingles (herpes zoster), diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

Accordingly, one aspect of the present invention is to provide the use of esters of capsaicin which can be incorporated into a topical formulation in high concentration for the treatment of post-herpetic neuralgia, shingles (herpes zoster), cold sores, diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

In some embodiments, the methods of the present invention neither destroy healthy, uninfected tissue nor result in any local or systemic side effects, scarring, disfigurement or discomfort to the subject treated. Furthermore, in some embodiments, the use of the esters of the present invention almost eliminates the occurrence of skin irritation and rashes unlike the free capsaicin.

In some embodiments of the methods of the invention, a topical formulation comprising esters of capsaicin in high concentration can be used at least once a day to the body surface containing post-herpetic neuralgia, shingles (herpes zoster), diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

There is further provided a method for the treatment of post-herpetic neuralgia, shingles (herpes zoster), cold sores, diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor, by the application of a cream, oil or a patch comprising either an ester of capsaicin or mixtures thereof, to the affected area of the subject's body.

There is also disclosed a method for treating post-herpetic neuralgia, shingles (herpes zoster), cold sores, diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor, said method involves the application of cream, oil or a patch comprising either an ester of capsaicin or mixtures thereof, to the affected area of a subject for a period of time and at a sufficient concentration to eradicate symptoms in the subject.

In some embodiments, the pharmaceutical compositions of the present invention can include one or more salicylates. Examples of salicylates include but not limited to salicylic acid, acetylsalicylate, methylsalicylate, methyl acetylsalicylate, trolamine salicylate and lysine salicylate. In some embodiments, the pharmaceutical compositions of the present invention can include one or more NSAIDs. The NSAIDs include but are not limited to salicylates such as aspirin (acetylsalicylic acid), diflunisal and salsalate; p-amino phenol derivatives such as paracetamol and phenacetin; propionic acid derivatives such as ibuprofen′ naproxen, fenoprofen, ketoprofen, dexketoprofen, flurbiprofen, oxaprozin and loxoprofen; acetic acid derivatives such as indomethacin, sulindac, etodolac, ketorolac, diclofenac and nabumetone; enolic acid (oxicam) derivatives such as piroxicam, meloxicam, tenoxicam, droxicam, lornoxicam and isoxicam; and fenamic acid derivatives (fenamates) such as mefenamic acid, meclofenamic acid, flufenamic acid and tolfenamic acid.

In some embodiments, the pharmaceutical compositions of the present invention can include one or more corticosteroids. Corticosteroids include but are not limited to alclometasone dipropionate, amcinonide, amcinafel, amcinafide, beclamethasone, betamethasone, betamethasone dipropionate, betamethasone valerate, budesonide, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone diacetate, dichlorisone, esters of betamethasone, flucetonide, flucloronide, fluorocortisone, flumethasone, flunisolide, fluocinonide, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, medrysone, meprednisone, methylprednisone, methylprednisolone, mometasone furoate, paramethasone, prednisone, prednisolone, prednidone, triamcinolone acetonide, and triamcinolone.

In some embodiments, the pharmaceutical compositions of the present invention can include one or more NMDA antagonists. Examples of NMDA antagonists include but are not limited to dextromethorphan and dextrorphan.

In some embodiments, the pharmaceutical compositions of the present invention can include one or more opioid agonists and/or antagonists. Examples of opioid agonists/antagonists include but are not limited to purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives

In some embodiments, the pharmaceutical compositions of the present invention can include one or more α2δ ligands. Examples of α2δ ligands include but are not limited to gabapentin and pregabalin.

In some embodiments, the pharmaceutical compositions of the present invention can include one or more agents selected from menthol, boswellic acid, DMSO, methyl sulfonylmethan, emu oil and hyaluronic acid.

In some embodiments, the pharmaceutical compositions of the present invention can include one or more agents selected from santalol, santalyl acetate, amyris alcohol and amyris acetate.

In some embodiments, the pharmaceutical compositions of the present invention can additionally include one or more pharmaceutically acceptable excipients. One of ordinary skill in the art would be familiar with pharmaceutically acceptable excipients. For example, the pharmaceutically acceptable excipient may be a water soluble sugar, such as mannitol, sorbitol, fructose, glucose, lactose, and sucrose.

In some embodiments, the pharmaceutical compositions of the present invention may further comprise one or more pharmaceutically acceptable antioxidants. Any pharmaceutically acceptable antioxidant known to those of ordinary skill in the art is contemplated for inclusion in the present pharmaceutical compositions. For example, the pharmaceutically acceptable antioxidant may be selected from the group consisting of ascorbic acid, sodium ascorbate, sodium bisulfate, sodium metabisulfate and monothio glycerol.

In some embodiments, the pharmaceutical compositions of the present invention may further comprise one or more pharmaceutically acceptable preservatives. Any pharmaceutically acceptable preservative known to those of ordinary skill in the art is contemplated for inclusion in the present pharmaceutical compositions. Examples of such preservatives include methylparaben, methylparaben sodium, propylparaben, propylparaben sodium, benzalkonium chloride, and benzthonium chloride.

In some embodiments, the pharmaceutical compositions of the present invention may further comprise one or more pharmaceutically acceptable buffering agents. Any pharmaceutically acceptable buffering agent known to those of ordinary skill in the art is contemplated for inclusion in the present pharmaceutical compositions. Examples of such buffering agents include of monobasic sodium phosphate, dibasic sodium phosphate, sodium benzoate, potassium benzoate, sodium citrate, sodium acetate, and sodium tartrate.

In some embodiments, the pharmaceutical compositions of the present invention may further comprise one or more pharmaceutically acceptable skin penetration enhancers. Examples of such skin penetration enhancers include but not limited to fatty alcohols such as decanol, lauryl alcohol, linolenyl alcohol, n-octanol and oleyl alcohol; fatty acid esters such as ethyl acetate, dodecyl N,N-dimethylamino acetate, glycerol monolaurate, glycerol monooleate, isopropyl myristate, methyl laurate and sorbitan monooleate; fatty acids such as lauric acid and oleic acid; biologics such as lecithin, amines and amides such as N,N-dimethyl-m-toluamide, lauryl-amine and urea; complexing agents such as cyclodextrin, hydroxypropyl methylcellulose and liposomes; surfactants such as Brij 36T, sodium lauryl sulfate and sorbitan monooleate; other compounds such as dimethyl isosorbide, bisabolol, eucalyptol, menthol, terpenes, N-methyl pyrrolidone, azone, DMSO, MSM, decylmethyl sulfoxide, dimethyl formamide, dimethyl acetamide, glycols and propylene glycol.

In some embodiments, the pharmaceutical compositions of the present invention can include a long chain ester of capsaicin or mixtures thereof in high concentration. For example, the concentration of long chain ester of capsaicin or mixtures thereof, can be from about 10% by weight to up to about 50% by weight. In some embodiments, the composition comprises about 14.25% of capsaicin palmitate by weight, which corresponds to about 8% of capsaicin content by weight. In some embodiments, the concentration of long chain ester of capsaicin or mixtures thereof corresponds to about 5% of capsaicin content by weight.

In some embodiments of the present invention, the pharmaceutical composition includes more than one of the esters of capsaicin set forth above. In other embodiments of the present invention, the pharmaceutical composition includes one or more secondary therapeutic agents directed to a disease or health-related condition, as discussed below.

The present invention also generally pertains to methods of treating or preventing a pathological condition in a subject, comprising providing a therapeutically effective amount of any of the pharmaceutical compositions set forth above, and administering the composition to the subject. The subject can be any subject, such as a mammal or avian species. In certain particular embodiments, the mammal is a human. The human may be an individual affected by or at risk of developing a disease or condition amenable to therapy with capsaicin. For example, the pathological condition may be post-herpetic neuralgia, shingles (herpes zoster), diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

In certain embodiments of the methods of the present invention, the method involves administering to the subject a therapeutically effective amount of a secondary agent. The secondary agent can be any pharmacologic agent known or suspected to be of benefit in the treatment or prevention of a disease or health-related condition in a subject. For example, in some embodiments, the secondary agent is a secondary pain relieving agent. Secondary pain relieving agents, which include morphine, are well-known to those of ordinary skill in the art. Examples of such agents include aspirin, acetaminophen (Tylenol) or other aspirin-like drugs called nonsteroidal anti-inflammatory drugs (NSAIDs), weak narcotics such as codeine (Tylenol with codeine), hydrocodone (Vicodin or Lortab), Percocet, Percodan or propoxyphene (Darvon), strong opioids such as morphine, Demerol, Dilaudid, fentanyl (duragesic patches) and methadone.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The chemical structures of capsaicins.

FIG. 2. Activation of TRPV1 by capsaicin results in sensory neuronal depolarization, and can induce local sensitization to activation by heat, acidosis, and endogenous agonists. Topical exposure to capsaicin leads to the sensations of heat, burning, stinging, or itching. High concentrations of capsaicin or repeated applications can produce a persistent local effect on cutaneous nociceptors, which is best described as defunctionalization and constituted by reduced spontaneous activity and a loss of responsiveness to a wide range of sensory stimuli.

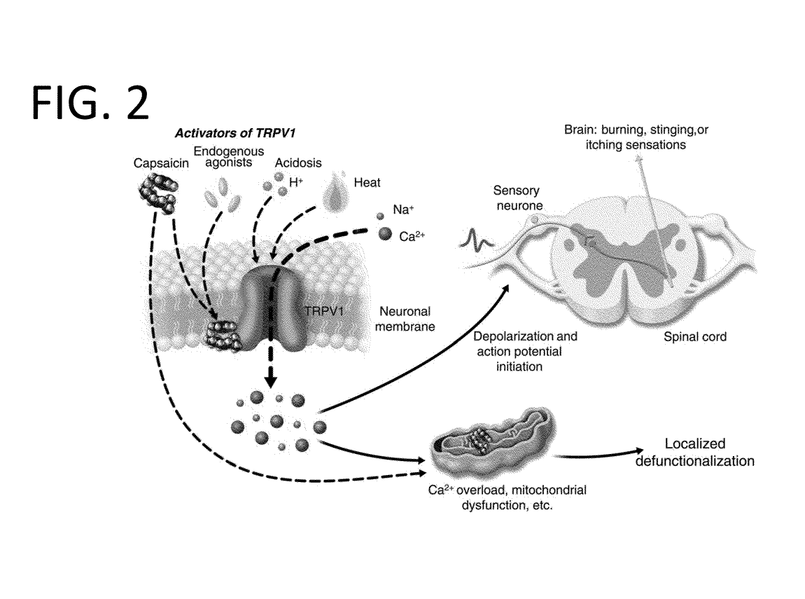

FIG. 3. Multiple mechanisms underlie capsaicin-induced defunctionalization. Inactivation of voltage-gated Na⁺ channels and direct pharmacological desensitization of plasma membrane TRPV1 receptors may contribute to an immediate reduction on neuronal excitability and responsiveness. More persistent effects may be due to the overwhelming of intracellular Ca²⁺ buffering capacity by extracellular Ca²⁺ entering through TRPV1 and being released from intracellular stores, with subsequent activation of calcium-dependent proteases and cytoskeleton breakdown. Microtubule depolymerization may interrupt fast axonal transport. At concentrations far in excess of those required to activate TRPV1, capsaicin can also render mitochondria dysfunctional by directly inhibiting electron chain transport. Thus mitochondria are a key convergence point for defunctionalization.

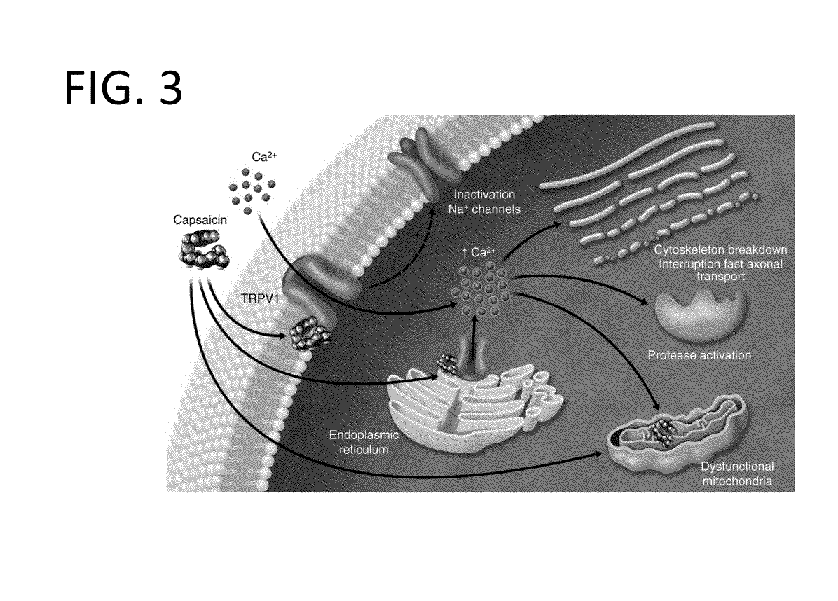

FIG. 4. The site of action of topical capsaicin is in the skin, and pain relief is not mediated by transdermal systemic delivery. Owing to near insolubility in water, capsaicin is not readily absorbed into the microvasculature. When cutaneous nociceptors are hypersensitive and sometimes spontaneously active, localized defunctionalization of capsaicin-responsive nerve fibre terminals in the epidermis and dermis can reduce the afferent barrage which may drive pain syndromes. Inset shows how mitochondrial dysfunction leads to nerve terminal retraction.

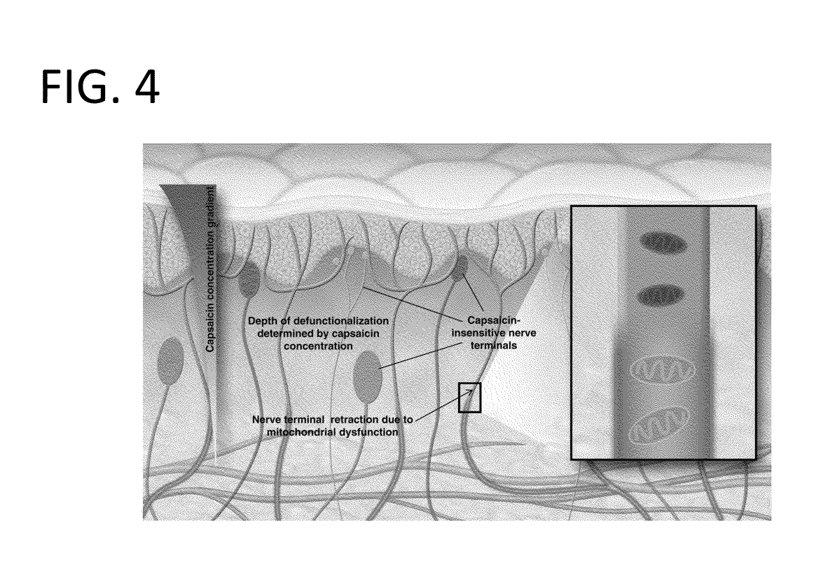

FIG. 5. Formula I: The chemical structures of capsaicin esters.

FIG. 6. Chemical structure of menthol.

FIG. 7. Chemical structure of beta-boswellic acid.

FIG. 8. Chemical structure of salicylic acid.

FIG. 9. Chemical structure of hydrocortisone.

FIG. 10. Chemical structure of dextromethorphan.

FIG. 11. Chemical structure of tramadol.

FIG. 12. Chemical structure of gabapentin.

FIG. 13. Chemical structure of santalol.

FIG. 14. Chemical structures of valerianol, eudesmol and elemol.

### DETAILED DESCRIPTION OF THE INVENTION

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

It must be noted that, as used in this specification, the singular forms “a,” “an” and “the” include plural referents unless the context clearly dictates otherwise. Thus, for example, reference to “a pharmacologically active agent” includes a combination of two or more pharmacologically active agents, and the like. In describing the present invention, the following terminology will be used in accordance with the definitions set out below.

As used herein in the claim(s), when used in conjunction with the word “comprising”, the words “a” or “an” may mean one or more than one. As used herein “another” may mean at least a second or more.

As used herein, the word “about” means±10% of the numerical value indicated.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned which also induce the desired pharmacologic effect.

The term “topical administration” is used in its conventional sense to mean delivery of a topical drug or pharmacologically active agent to the skin or mucosa.

“Carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

By an “effective” amount of a drug or pharmacologically active agent is meant a nontoxic but sufficient amount of the drug or agent to provide the desired effect.

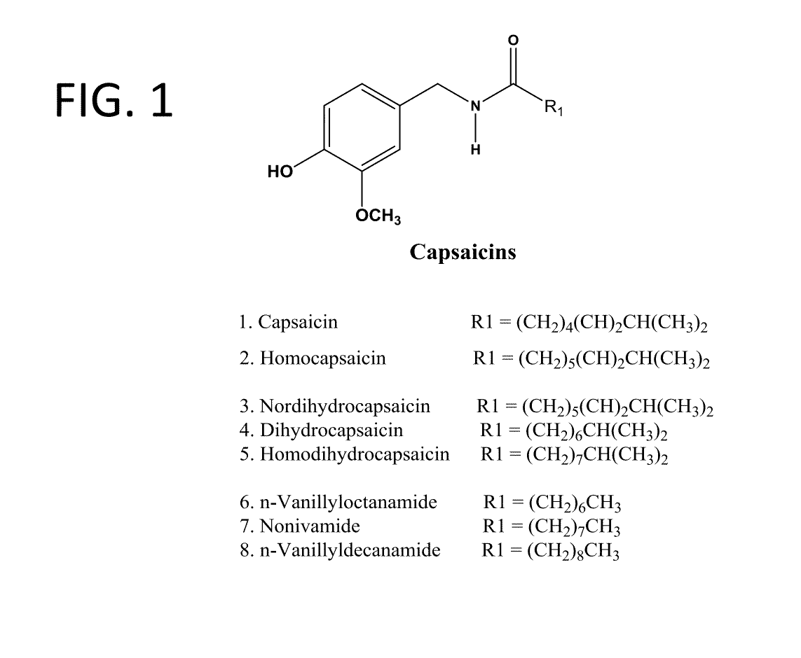

The term “capsaicin” or “capsaicins” as used herein is intended to encompass the compounds shown in FIG. 1 and any mixture thereof.

The term “long chain” as used herein is intended to encompass the esters of capsaicin wherein the R-group contains at least 11 carbon atoms.

The term “high concentration” as used herein is intended to encompass the topical composition containing esters of capsaicin which are at least about 5% equivalent of capsaicin content by weight.

The term “santalol” as used herein is intended to encompass not only α- and β-santalol, but any isomer or any compounded mixture thereof.

The term “santalyl acetate” as used herein is intended to encompass not only α- and β-santalyl acetate, but any isomer or any compounded mixture thereof.

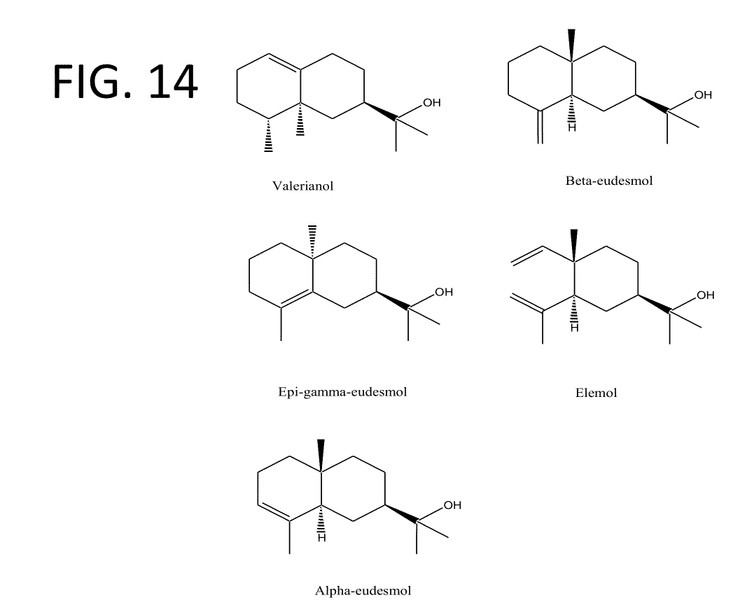

The term “amyris alcohol” used as herein refers to the alcohol obtained from amyris oil by removing the volatile hydrocarbons, such as, for example, under vacuum. The total sesquiterpene tertiary alcohol content in amyris alcohol varies from about 60% to about 85%. The major constituents of amyris alcohol are isomeric compounds, eudesmol, valerianol and elemol. The chemical formula for eudesmol, valerianol and elemol is C₁₅H₂₆O and the chemical structures are shown in FIG. 14.

The term “amyris acetate” as used herein is intended to encompass the fully acetylated amyris alcohol.

The compositions comprising long chain ester derivatives of capsaicin of the present invention have utility over capsaicin and existing derivatives. For example, in view of their high lipophilicity, non-irritation to the skin, almost non-burning sensation at the site of application and stability, these ester derivatives are highly desirable for topical administration in high concentration as compared to capsaicin. In addition, because of their stability and non-toxic nature, these agents can be made more readily available to the general public.

The inventors have surprisingly and unexpectedly discovered that compositions containing high concentration of long chain ester derivatives of capsaicin have therapeutic utility in treating pain in humans as they are almost non-burning to skin without the loss of efficacy. These compositions thus provide for a novel form of therapy of any disease or condition wherein capsaicin is believed to be of benefit, including but not limited to, post-herpetic neuralgia, shingles (herpes zoster), fever blister, cold sores, diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

### A. CAPSAICIN ESTERS OF THE PRESENT INVENTION

The present invention generally pertains to pharmaceutical compositions containing a compound of formula (I):

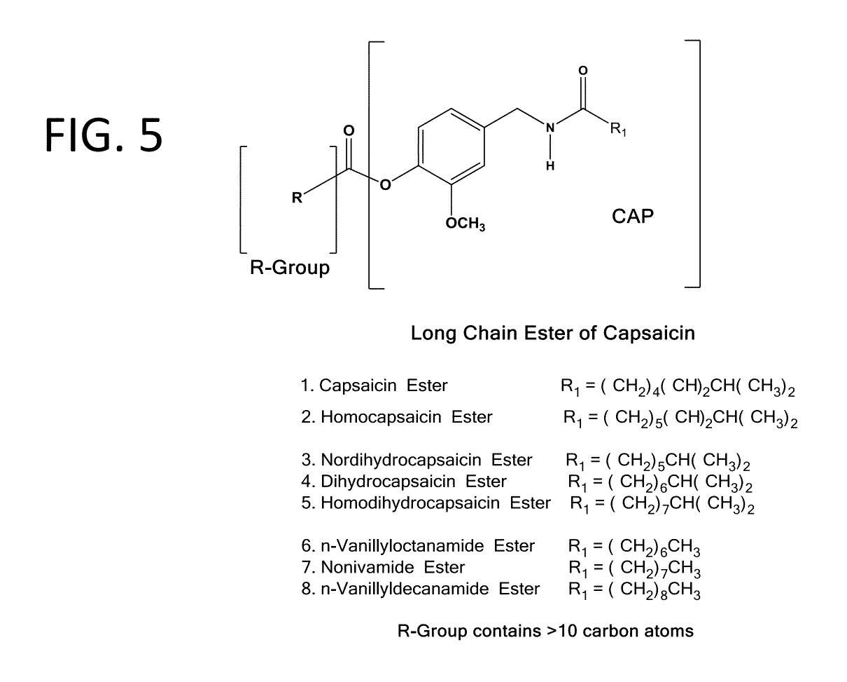

R—CO-CAP (I)

wherein CAP refers to the capsaicin group represented in FIG. 5.

In formulae I, R is selected from alkyl groups of ranging from 11 up to about 22 carbon atoms and aryl groups of ranging from 11 up to about 22 carbon atoms and alkylene group of ranging from 11 up to about 22 carbon atoms and an arylene group of ranging from 11 up to about 22 carbon atoms. The alkyl, aryl and alkylene groups may be substituted or unsubstituted, branched or straight chains. In addition, R may contain heteroatoms and may be straight chained or branched.

Examples of suitable straight-chain alkyl groups in formula I include 1-hendecyl, 1-pentadecyl, 1-heptadecyl, 1-hexadecyl, 1-octadecyl and the like groups.

In some embodiments, R has 11, 12, 13, 14, 15, 16, 17, 18, 19, 20, 21 or 22 carbon atoms.

Among the compounds represented by the general Formula I, in some embodiments, R is one of the following groups: 1-hendecyl, 1-pentadecyl, 1-heptadecyl, 1-hexadecyl and 1-octadecyl.

The compounds of Formula I are long chain esters of capsaicin which can be incorporated into a topical formulation in high concentration for treating diseases such as post-herpetic neuralgia, shingles (herpes zoster), diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

### B. METHODS OF SYNTHESIS OF HIGH PURITY ESTER OF CAPSAICIN

The compounds used in the present invention can be prepared by any method known to those of ordinary skill in the art. One method that has been utilized for efficient preparation of the ester of capsaicin of the present invention is through dissolution of the compound in methylene dichloride. Since capsaicin USP contains >95% of capsaicins, to this solution slightly in excess of 1.1 mole equivalent of anhydrous triethylamine is added with stirring at room temperature. To this solution mole equivalent of an acid chloride is added with stirring while keeping the temperature around 25° C. After that, the solution was refluxed for 2-5 hours and stirred for 12-17 hours at room temperature. The reaction mixture was then washed with equal amount of water three to four times to remove the unreacted amine and its salt in a separating funnel. The organic phase was washed 3-4 times with dilute hydrochloric acid solution in a separating funnel to remove any amine present in the organic solution. The reaction mixture was then washed with equal amount of 10% sodium bicarbonate solution three to four times to remove the acid and salts in a separating funnel. The reaction mixture was then washed with equal amount of water three to four times in a separating funnel until the washed out solution has neutral pH. The organic phase was dried with anhydrous sodium sulfate overnight and the methylene dichloride was removed in a rotary evaporator under vacuum. The resultant wax like material is called the ester capsaicin as all of the capsaicinoids present is converted into the corresponding ester.

In some embodiments, in order to incorporate the ester of capsaicin in high concentration in topical formulations, it is essential to remove residual capsaicinoids from the esters. While the purification can be achieved by preparative column chromatography, the inventors have developed a process by which the wax like esters can be purified by repeated re-crystallization in a suitable solvent. The following method was developed to obtain the esters of capsaicin in high purity having less than 0.001% of capsaicinoids.

The capsaicin palmitate was thoroughly dried and then weighed, (W_{CP}), in grams. This weight was multiplied by 1.21 to obtain the total volume when the capsaicin palmitate is dissolved in methanol, and this quantity was noted as the dissolved volume (D_{V}). The amount of methanol, X, in milliliters, to be added to make a 4% solution was calculated by the equation below:

$$X = \frac{{W}_{CP}}{0.04} - {D}_{V} \qquad ( II )$$

The weighed capsaicin palmitate was transferred into a suitable container and the calculated amount of methanol was added to it. The solution was stirred while applying low heat to the container in a water bath; the solids were completely dissolved either under mild to high shear stirring between 25° C. to 35° C., or under low shear stirring between 35° C. to 45° C.

The solution was filtered through a 11 μm or smaller filter media to remove any dust or insoluble particles and this clean solution was transferred into a crystallization vessel (such as a cylinder) and then the top was covered with laboratory film and aluminum foil. The vessel was refrigerated at 4° C. and it was kept there for 8-12 hours, or until no more precipitate formed. The precipitate was removed and the supernatant was transferred to a suitably sized Buchner funnel fitted with Grade 2 or 3 qualitative filter paper. The filtered solution was collected under vacuum and set aside. The filter cake was washed with methanol that has been cooled to 4° C., using approximately 1 L of methanol for every 250 g of capsaicin palmitate that was initially dissolved. The washed solution was collected under vacuum and combined with the solution that was collected earlier and set aside. The filter cake was dried over vacuum until no more solution was coming over into the collection flask. The cake was transferred and evenly divided in pyrex glass trays and then placed the trays into an vacuum oven. The capsaicin palmitate was dried under high vacuum at 35° C. for 12 hours, or until there is no more methanol odor and the material is dry and crumbled to the touch.

The recrystallized capsaicin palmitate was weighed, recording the weight (W_{obtained}) in grams. The remaining capsaicin palmitate in the filtrate solution (W_{remaining}) was calculated as,

W_{CP}−W_{obtained}=W_{remaining}.

The excess solvent was removed from the filtrate using a rotary evaporator so that the volume of the solvent left would satisfy the equation II. The concentrated solution was refrigerated at 4° C. as described in the previous paragraph to obtain the crystallized capsaicin palmitate and processed as described in the previous paragraph. The crystallized capsaicin palmitate from the two steps was combined together and the yield was calculated.

### C. OTHER AGENTS

In some embodiments, the esters of capsaicin of the present invention can be incorporated into pharmaceutical compositions comprising other pain relieving agents such as salicylates, menthol, boswellic acids, DMSO, methyl sulfonylmethane, NSAIDs, corticosteroids, emu oil, opioid agonists and antagonists, NMDA antagonists, tramadol, hyaluronic acid, α2δ ligands, aloe vera gel and aloe vera juice.

In addition, esters of capsaicin of the present invention can be incorporated into pharmaceutical compositions comprising santalol, santalol acetate, amyris alcohol or amyris acetate to improve the therapeutic efficacy in treating fever blisters and cold sores.

A non-limiting description of other agents or class of other agents is described below.

### 1). Menthol

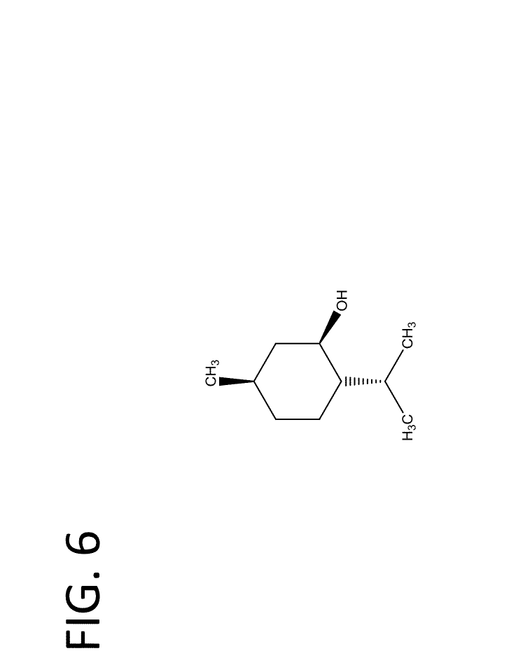

Menthol (FIG. 6) is a waxy, crystalline substance, clear or white in color, which is solid at room temperature and melts slightly above. The main form of menthol occurring in nature is (−)-menthol, which is assigned the (1R,2S,5R) configuration. Menthol has local anesthetic and counterirritant qualities, and it is widely used to relieve minor throat irritation. Menthol also acts as a weak kappa-opioid receptor agonist.

Menthol's ability to chemically trigger the cold-sensitive TRPM8 receptors in the skin is responsible for the well-known cooling sensation it provokes when inhaled, eaten, or applied to the skin (Eccles R, Menthol and Related Cooling Compounds, J. Pharm. Pharmacol., 1994; 46 (8): 618-630). In this sense, it is similar to capsaicin, which stimulates heat sensors, also without causing an actual change in temperature

Menthol's analgesic properties are mediated through a selective activation of K-opioid receptors (Galeottia, et al., Menthol: a natural analgesic compound, Neuroscience Letters 2002; 322 (3): 145-148). Menthol also blocks voltage-sensitive sodium channels, reducing neural activity that may stimulate muscles (Haeseler, et al., Voltage-dependent block of neuronal and skeletal muscle sodium channels by thymol and menthol, European Journal of Anaesthesiology 2002; 19 (8): 571-579). Menthol also enhances the efficacy of ibuprofen in topical applications via vasodilation, which reduces skin barrier function (Braina, et al., The Role of Menthol in Skin Penetration from Topical Formulations of Ibuprofen 5% in vivo, Skin Pharmacol Physiol, 2006; 19:17-21).

### 2). Boswellic Acid

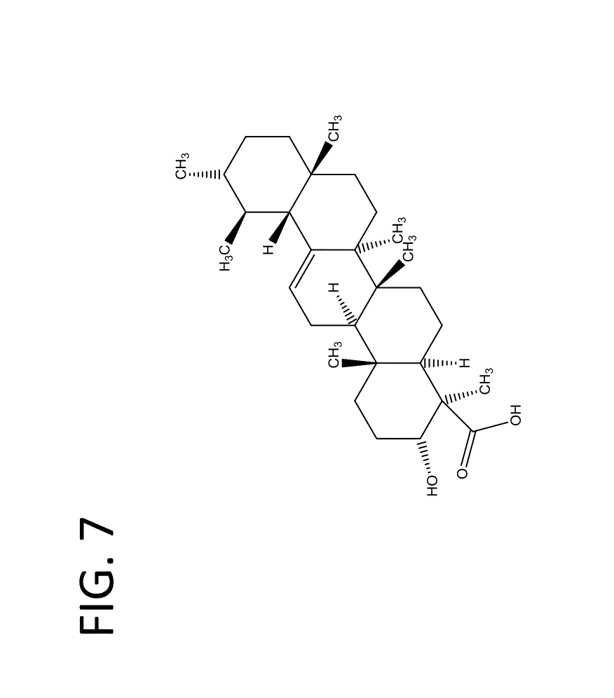

In Ayurvedic medicine, Indian frankincense (Boswellia serrata) has been used for hundreds of years for treating arthritis. The extract of the Boswellia serrata contains Boswellic acid and other pentacyclic triterpene acids and Beta-boswellic acid (FIG. 7) is the major constituent. Extracts of Boswellia serrata have been clinically studied for osteoarthritis and joint function, particularly for osteoarthritis of the knee. Positive effects of Boswellia in some chronic inflammatory diseases including rheumatoid arthritis, bronchial asthma, osteoarthritis, ulcerative colitis and Crohn's disease have been reported (Ammon H P., Modulation of the immune system by Boswellia serrata extracts and boswellic acids. Phytomedicine. 17(11):862-7, 2010 September). Extract of Boswellia serrata has anti-Inflammatory and anti-arthritic property that can reduce the pain and inflammation of the joints of the body (Kimmatkar N, et al.; Efficacy and tolerability of Boswellia serrata extract in the treatment of osteoarthritis of knee—a randomized double blind placebo controlled study. Phytomedicine 2003; 10 (1): 3-7). Animal studies performed in India show ingestion of a defatted alcoholic extract of Boswellia decreased polymorphonuclear leukocyte infiltration and migration, decreased primary antibody synthesis and almost totally inhibited the classical complement pathway (Sharma M L, et al., Anti-arthriticactivity of boswellic acids in bovine serum albumin (BSA)-induced arthritis. Int J Immunopharmacol 1989; 11:647-652).

### 3). Methylsulfonylmethane

Methylsulfonylmethane (MSM) is an organosulfur compound with the formula (CH₃)₂SO₂. It occurs naturally in some primitive plants, is present in small amounts in many foods and beverages, and is marketed as a dietary supplement. MSM is sold as a dietary supplement and marketed with a variety of claims, often in combination with glucosamine and/or chondroitin for helping to treat or prevent osteoarthritis. Small-scale studies of possible treatments with MSM have been conducted on both animals and humans. These studies of MSM have suggested some benefits, particularly for treatment of osteoarthritis. A review by Brien, et al., (Systematic review of the nutritional supplements dimethyl sulfoxide (DMSO) and methylsulfonylmethane (MSM) in the treatment of osteoarthritis, Osteoarthritis and Cartilage, 2008; 16:1277) of the two small randomized controlled trials of methylsulfonylmethane in osteoarthritis knee pain relief reported significant improvement in pain outcomes in the treatment group compared to comparator treatments. After several reports that MSM helped arthritis in animal models, one study by Usha et al. (Double-blind, parallel, placebo-controlled study of oral glucosamine, methylsulfonylmethane and their combination in osteoarthritis, Clinical Drug Investigation, 2004; 24:353-63) had confirmed that 1.5 g per day MSM (alone or in combination with glucosamine sulfate) was helpful in relieving symptoms of knee osteoarthritis.

### 4). Salicylic Acid and its Derivatives

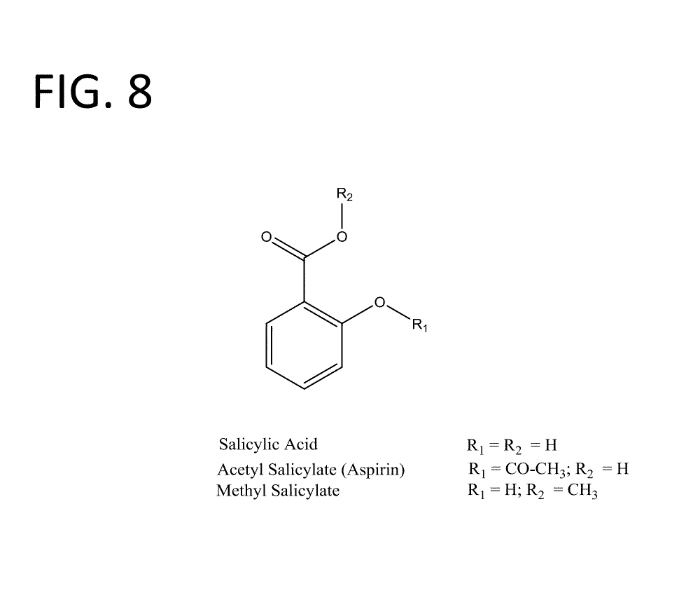

Salicylic acid (FIG. 8) is known for its ability to ease aches and pains and reduce fevers. These medicinal properties, particularly fever relief, have been known since ancient times, and it was used as an anti-inflammatory drug. In modern medicine, salicylic acid and its derivatives are used as constituents of some rubefacient products. For example, methyl salicylate is used as a liniment to soothe joint and muscle pain, and choline salicylate is used topically to relieve the pain of aphthous ulcers. Aspirin, also known as acetylsalicylic acid, is a salicylate drug, often used as an analgesic to relieve minor aches and pains, as an antipyretic to reduce fever, and as an anti-inflammatory medication.

High concentration (17%) of salicylic acid liquid formulation is used on the skin to treat common skin and foot (plantar) warts. Salicylic acid helps cause the wart to gradually peel off. This product is also used to help remove corns and calluses. This product should not be used on the face or on moles, birthmarks, warts with hair growing from them, or genital/anal warts. Salicylic acid is a keratolytic. It belongs to the same class of drugs as aspirin (salicylates). It works by increasing the amount of moisture in the skin and dissolving the substance that causes the skin cells to stick together. This makes it easier to shed the skin cells. Warts are caused by a virus. Salicylic acid does not affect the virus.

Methyl salicylate is used as a rubefacient in deep heating liniments (such as Bengay ointment), and in small amounts as a flavoring agent at no more than 0.04%. It is also used to provide fragrance to various products and as an odor-masking agent for some organophosphate pesticides. If applied in too high quantities it can cause stomach and kidney problems. Methyl salicylate is one of several antiseptic ingredients in Listerine mouthwash produced by the Johnson & Johnson company. It is also used in the “Dencorub Extra Strength” heat cream, which is used to treat joint and muscular pain and is a product of the Dencorub company.

Trolamine salicylate is the salt formed between triethanolamine and salicylic acid. It is used as an ingredient in sunscreens, analgesic creams, and cosmetics. The salicylic acid portion contributes to both the sun protection effect (by absorbing UVB rays) and to the analgesic effect. The triethanolamine neutralizes the acidity of the salicylic acid. One benefit of this topical analgesic is that it has no odor, in contrast to other topical analgesics such as menthol.

Intravenous lysine acetylsalicylate, the salt formed between lysine and aspirin, has been shown to be effective in the treatment of acute migraine attacks (Weatherall et al., Intravenous aspirin (lysine acetylsalicylate) in the inpatient management of headache, Neurology 2010: 75:1098-1103).

### Nonsteroidal Anti-inflammatory Drugs (NSAIDs)

Non-narcotic analgesics, also known as non-steroidal anti-inflammatory drugs (NSAID), are widely administered orally in the treatment of inflammation and mild to moderate pain. Within this class, the compounds vary widely in their chemical structure and in their biological profiles as analgesics, anti-inflammatory agents and antipyretic agents. The term “nonsteroidal” is used to distinguish these drugs from steroids, which, among a broad range of other effects, have a similar eicosanoid-depressing, anti-inflammatory action. As analgesics, NSAIDs are unusual in that they are non-narcotic. The most prominent members of this group of drugs are aspirin, ibuprofen, and naproxen, all of which are available over the counter in many areas.

Most NSAIDs act as nonselective inhibitors of the enzyme cyclooxygenase (COX), inhibiting both the cyclooxygenase-1 (COX-1) and cyclooxygenase-2 (COX-2) isoenzymes. COX catalyzes the formation of prostaglandins and thromboxane from arachidonic acid (itself derived from the cellular phospholipid bilayer by phospholipase A₂). Prostaglandins act (among other things) as messenger molecules in the process of inflammation.

The NSAIDs include but not limited to salicylates such as aspirin (acetylsalicylic acid), diflunisal and salsalate; p-amino phenol derivatives such as paracetamol and phenacetin; propionic acid derivatives such as ibuprofen^{l}, naproxen, fenoprofen, ketoprofen, dexketoprofen, flurbiprofen, oxaprozin and loxoprofen; acetic acid derivatives such as indomethacin, sulindac, etodolac, ketorolac, diclofenac and nabumetone; enolic acid (oxicam) derivatives such as piroxicam, meloxicam, tenoxicam, droxicam, lornoxicam and isoxicam; and fenamic acid derivatives (fenamates) such as mefenamic acid, meclofenamic acid, flufenamic acid and tolfenamic acid.

Apart from the anti-inflammatory properties, the NSAIDs have been tested for controlling angiogenesis. For example, U.S. Pat. No. 5,847,002 discloses the use of: (a) a non-steroidal anti-inflammatory agent, and (b) hyaluronic acid, in the manufacture of a pharmaceutical composition for inhibiting, controlling and/or regressing angiogenesis in a therapy.

### 5). Topical Steroids

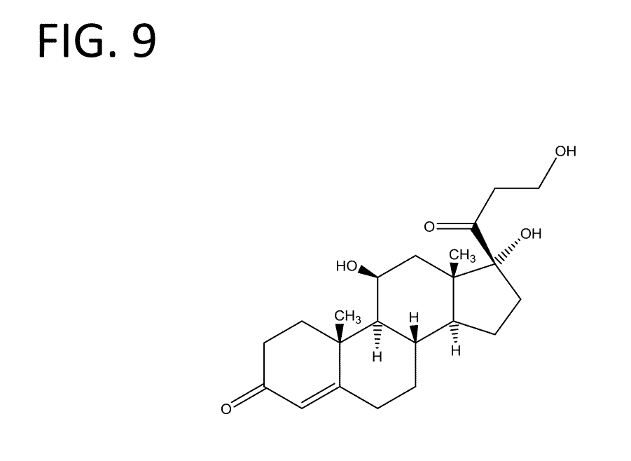

Topical steroids are the topical forms of corticosteroids (FIG. 9). Topical steroids are the most commonly prescribed topical medications for the treatment of rash, eczema, and dermatitis. Topical steroids have anti-inflammatory properties, and are classified based on their vasoconstriction abilities (Habif, (1990). Clinical dermatology: a color guide to diagnosis and therapy (2nd ed.). St. Louis: Mosby. pp. 27-30). There are numerous topical steroid products. All the preparations in each class have the same anti-inflammatory properties, but essentially differ in base and price.

Topical corticosteroids are useful for their anti-inflammatory, anti-pruritic and vasoconstrictive actions. Corticosteroids (or corticoids) are any steroids (lipids that contain a hydrogenated cyclopentoperhydrophenanthrene ring system) elaborated by the adrenal cortex (except sex hormones of adrenal origin) in response to the release of adrenocorticotrophin or adrenocorticotropic hormone by the pituitary gland, or to any synthetic equivalent, or to angiotensin II. Corticosteroids include but are not limited to alclometasone dipropionate, amcinonide, amcinafel, amcinafide, beclamethasone, betamethasone, betamethasone dipropionate, betamethasone valerate, budesonide, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone diacetate, dichlorisone, esters of betamethasone, flucetonide, flucloronide, fluorocortisone, flumethasone, flunisolide, fluocinonide, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, medrysone, meprednisone, methylprednisone, methylprednisolone, mometasone furoate, paramethasone, prednisone, prednisolone, prednidone, triamcinolone acetonide, and triamcinolone.

Hydrocortisone was the first corticosteroid found to be topically effective. Other more potent glucocorticoids, which are a subset of corticosteroids that affect carbohydrate metabolism, inhibit corticotropin secretion, and possess pronounced anti-inflammatory activity, have since been developed. Currently, topical steroids are among the most frequently prescribed of all dermatological drug products.

It is believed that glucocorticoids exert their potent anti-inflammatory effects by inhibiting the formation of prostaglandins and other derivatives of the arachidonic acid pathway. It is known that glucocorticoids inhibit the release of phospholipase A2, the enzyme responsible for liberating arachidonic acid from cell membranes, thus inhibiting the arachidonic acid pathway. Currently, it is believed that glucocorticoids inhibit phospholipase A2, in cells by directly inducing phosphorylation of the enzyme.

Steroids are commonly divided into two classes, fluorinated and nonfluorinated. Fluorinated steroids have been chemically modified to increase potency. These modifications, such as halogenation and methylation, can result in improved activity within the target cell and in decreased breakdown to inactive metabolites. These modifications can also lead to more systemic side effects. However, modification of the chemical structure of the steroid is not the only way to increase potency.

The potency of topical steroid preparations is strongly correlated to their absorption through the skin (Megrab, et al., Oestradiol permeation through human skin and silastic membrane: effects of propylene glycol and supersaturation, Journal of Controlled Release (1995), vol. 36, No. 3, pp. 277-294). Treatment of the skin prior to application of the topical steroid may also affect the absorption of the compounds into the skin. Treatments with keratolytics or with fat solvents (such as acetone) disrupt the epidermal barrier and increase penetration. Hydrating the skin has also been shown to increase the penetration of the corticosteroids.

Once absorbed through the skin, topical corticosteroids are handled through pharmacokinetic pathways similar to systemically administered corticosteroids. The potencies of corticosteroids vary greatly and it is a challenge to increase the potency of any particular steroid (Bennett et al., “Optimization of bioavailability of topical steroids: non-occluded penetration enhancers under thermodynamic control,” Journal of Pharmacy and Pharmacology, vol. 37, No. 5, 1985, pp. 298-304).

### 6. Emu Oil

Emu oil is rendered from the fat of the emu, Dromaius novaehollandiae, a bird native to Australia. Emu oil and eucalyptus oil have been used historically by the Australian aborigines for the treatment of fevers, coughs, arthritic joints, bruises, cuts and sores (Whitehouse M W, et al., Emu oil(s): A source of non-toxic transdermal anti-inflammatory agents in aboriginal medicine, Inflammopharmacology, 1998; 6 (1): 1-8).

Pure emu oil can vary widely in color and viscosity, but, assuming the emu has enjoyed a natural diet, is generally a yellow liquid.^{[5]} It is composed of approximately 70% unsaturated fatty acids. The largest component is oleic acid, a mono-unsaturated omega-9 fatty acid. Emu oil also contains about 20% linoleic acid (an omega-6 fatty acid) and 1-2% linolenic acid (an omega-3 fatty acid). A handful of studies have suggested that emu oil, applied topically, may have anti-inflammatory properties or promote wound healing in various rodent models. Emu oil is marketed and promoted as a dietary supplement with a wide variety of claimed health benefits.

### 7). Opioid Agonists and Antagonists

Opiates, a class of centrally acting compounds, are the most frequently used agents for pain control. Opiates are narcotic agonistic analgesics and are drugs derived from opium, such as morphine, codeine, and many synthetic congeners of morphine, with morphine and hydrocodone preparations being the most widely used opiates. Opiates are natural and synthetic drugs with morphine-like actions. Opiates are narcotic agonistic analgesics which produce drug dependence of the morphine type and are subject to control under Federal narcotics law and the laws of most other nations and international organizations because of their addicting properties and the subsequent destructive toll exacted on the abusers and those with any connection to them. The term “opiates” also includes opiate antagonists that are essentially devoid of agonist activity at any opiate receptor, partial agonists, and opiates with mixed actions, that is they are mixed function agonist-antagonists, which are agonists at some receptors and antagonists at other receptors.

The chemical classes of opiates with morphine like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives. The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine mesylate. The currently used morphinan derivative is levorphanol. The diphenyl-heptane derivatives include methadone and its congeners, and propoxyphene. Propionanilide derivatives include fentanyl citrate and its congeners sufentanil citrate and alfentanil hydrochloride. These opiate analgesics are discussed in detail in Goodman and Gilman's The Pharmacological Basis of Therapeutics, Chapter 21, “Opiate Analgesics and Antagonists”, pp. 485-521 (8^{th} ed. 1990), which is incorporated herein by reference.

The potency of all opiates is roughly comparable and can be effective against the most severe pain with appropriate dosing at intervals. However, all opiates have a wide variety of side effects that can decrease their clinical utility in certain situations. The side effects associated with the use of opiates include respiratory depression, reduced cough reflex, bronchial spasms, nausea, vomiting, release of histamine, peripheral vasodilation, orthostatic hypotension, alteration of vagal nerve activity of the heart, hyperexcitability of smooth muscles (sphincters), reduction of peristaltic motility in the gastrointestinal tract and urinary retention. Opiates also stimulate the release of adrenaline, anti-diuretic hormone, cause changes in the regulation of body temperature and sleep pattern, and are liable to promote the development of tolerance and addiction.

Furthermore, higher doses of agonistic-antagonistic analgesic agents are often associated with unpleasant sympathomimetic side effects such as tachycardia, increase in blood pressure, seizure and psychotomimetic effects such as drug induced psychosis, hyper-aggressive behavior and agitation. However, the risk of respiratory depression also decreases proportionately with the diminished analgesic activity of the higher doses. Agonistic-antagonistic analgesic agents with pharmacological activity similar to the morphine like opiates include pentazocine, nalbuphine, butorphanol, nalorphine, buprenorphine (a partial agonist), meptazinol, dezocine, and cyclazocine.

### 8). NMDA Antagonists

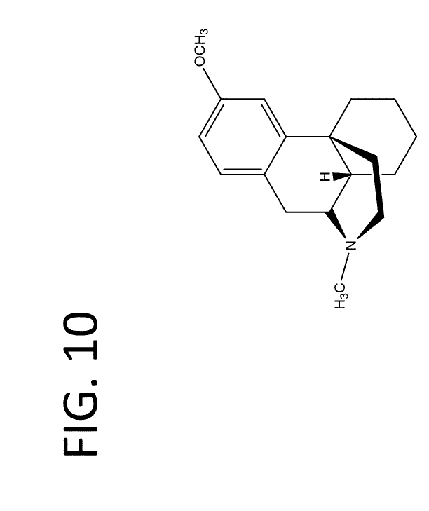

Dextromethorphan (frequently abbreviated as DM) is the common name for (+)-3-methoxy-N-methylmorphinan (FIG. 10). It is widely used as a cough suppressant, and is described in references such as Rodd (Rodd E H. Chemistry of Carbon Compounds, Elsevier Publ, New York, 1960) and Goodman and Gilman's Pharmacological Basis of Therapeutics (Brunton L L, et al., Goodman & Gilman's The Pharmacological Basis of Therapeutics. 12th ed. New York: McGraw-Hill, 2011. ISBN 13:978-0-07-1624428). Briefly, DM is a non-addictive opiate comprising a dextrorotatory enantiomer (mirror image) of the morphinan ring structure that forms the molecular core of most opiates. DM acts at a class of neuronal receptors known as sigma (σ) receptors. These are often referred to as p opiate receptors, but there is some question as to whether they are opiate receptors, so many researchers refer to them simply as p receptors, or as high-affinity dextromethorphan receptors. They are inhibitory receptors, which mean that their activation by DM or other μ-agonists causes the suppression of certain types of nerve signals. Dextromethorphan also acts at another class of receptors known as N-methyl-D-aspartate (NMDA) receptors, which are one type of excitatory amino acid (EAA) receptor. Unlike its agonist activity at p receptors, DM acts as an antagonist at NMDA receptors, which means that DM suppresses the transmission of nerve impulses mediated by NMDA receptors. Since NMDA receptors are excitatory receptors, the activity of DM as a NMDA antagonist also leads to the suppression of certain types of nerve signals, which may also be involved in some types of coughing. Due to its activity as a NMDA antagonist, DM and one of its metabolites, dextrorphan, are being actively evaluated as possible treatments for certain types of excitotoxic brain damage caused by ischemia (low blood flow) and hypoxia (inadequate oxygen supply), which are caused by events such as stroke, cardiac arrest, and asphyxia.

The anti-excitotoxic activity of dextromethorphan and dextrorphan, and the blockade of NMDA receptors by these drugs, are discussed in items such as Choi (Dextrorphan and dextromethorphan attenuate glutamate neurotoxicity. Brain Res 1987; 403: 333-6), Wong et al., (Dextrorphan and dextromethorphan, common antitussives, are antiepileptic and antagonize N-methyl-D-aspartate in brain slices. Neurosci Lett. 1988 Feb. 29; 85(2):261-266) and Steinberg et al., (Delayed treatment with dextromethorphan and dextrorphan reduces cerebral damage after transient focal ischemia, Neurosci Letters 1988; 89: 193-197) and U.S. Pat. No. 4,806,543. Dextromethorphan has also been reported to suppress activity at neuronal calcium channels (Carpenter C L et al., Dextromethorphan and dextrorphan as calcium channel antagonists, Brain Research 1988; 439: 372-375). Dextromethorphan and the receptors it interacts with are further discussed in Tortella et al., (Dextromethorphan and neuromodulation: old drug coughs up new activities. Trends Pharmacol Sci. 1989 December; 10(12):501-507) and Musacchio et al., (High affinity dextromethorphan binding sites in the guinea pig brain, J Pharmacol Exp Ther 1988; 247: 424-431).

DM disappears fairly rapidly from the bloodstream (see, e.g., Vettican S J et al, Phenotypic differences in dextromethorphan metabolism, Pharmaceut Res 1989; 6: 13-19). DM is converted in the liver to two metabolites called dextrorphan and 3-methoxymorphinan, by an enzymatic process called O-demethylation; in this process, one of the two pendant methyl groups is replaced by hydrogen. If the second methyl group is removed, the resulting metabolite is called 5-hydroxymorphinan. Dextrorphan and 5-hydroxymorphinan are covalently bonded to other compounds in the liver (primarily glucuronic acid or sulfur-containing compounds such as glutathione) to form glucuronide or sulfate conjugates which are eliminated fairly quickly from the body via urine bloodstream.

Dextrorphan, the major metabolite of the anti-tussive dextromethorphan, and ketamine, are known NMDA receptor antagonists. Unlike MK 801 they have few, if any, neurotoxic side effects. U.S. Pat. No. 5,352,683 discloses a method for the alleviation of chronic pain in a mammal suffering there from by administration of a nontoxic N-methyl-D-aspartate receptor antagonist such as dextromethorphan, dextrorphan, ketamine or pharmaceutically acceptable salt thereof, alone or in combination with a local anesthetic and optionally in sustained release dosage form.

In summary, Dextromethorphan and its active metabolite dextrorphan bind to the N-Methyl-D-Aspartate (NMDA) glutamate and nicotine/neuronal nicotinic receptors as inhibitors. Dextromethorphan and dextrorphan also bind to the receptor-gated (NMDA receptor mediated) and voltage-gated calcium channels, and the voltage-gated sodium channels as a blocker. Through these bindings, dextromethorphan and dextrorphan modulates the glutamate pathway in the central nervous system (CNS) and modulate most of the excitatory synaptic transmission. Dextromethorphan and dextrorphan also bind to the sigma receptors which are found in high concentrations in limbic and motor areas of the CNS sensory processing such as the dorsal root ganglia and the nucleus tractus solitarus (NTS). In addition, Dextromethorphan inhibits the reuptake of 5-HT (serotonin) and norepinephrine, thus modulating the monamine pathways.

### 9. Tramadol

Tramadol has the chemical name (+/−)-trans (RR,SS)-2-[(di-methylamino)methyl]-1-(3-methoxyphenyl)cyclohexanol, and which is often erroneously referred to in literature as the cis(RS,SR)diastereomer. Tramadol is a centrally acting, binary analgesic that is neither opiate-derived, nor is it an NSAID. It is used to control moderate pain in chronic pain settings, such as osteoarthritis and post-operative analgesia, and acute pain, such as dental pain.

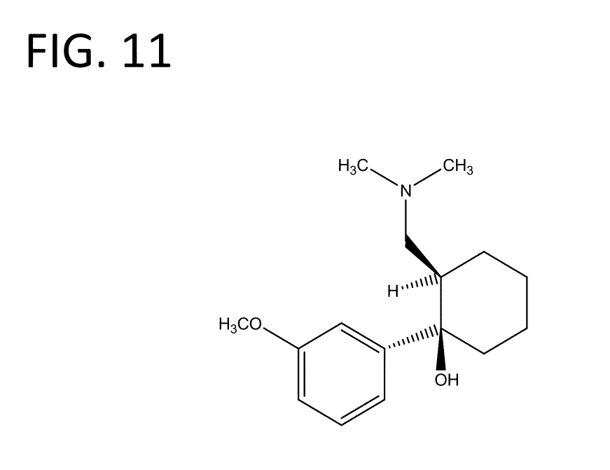

Tramadol is a racemate and consists of equal quantities of (+)- and (−)-enantiomers (FIG. 11). It is known that the pure enantiomers of tramadol have a differing pharmaceutical profiles and effects when compared to the racemate. The (+)-enantiomer is distinguished by an opiate-like analgesic action due its binding with the μ-opiate receptor, and both enantiomers inhibit 5-hydroxytryptamine (serotonin) and noradrenaline (norepinephrine) reuptake, which is stronger than that of racemic mixtures of tramadol, while distinct inhibition of noradrenaline reuptake is observed with the (−)-enantiomer. It has been proven for (+)- and (−)-tramadol that, depending upon the model, the two enantiomers mutually reinforce and enhance their individual actions (Raffa R B, Friderichs E, Reimann W, Shank R P, Codd E E, Vaught J L, Jacoby H I, Selve N. Complementary and synergistic antinociceptive interaction between the enantiomers of tramadol J Pharmacol Exp Ther 1993; 267: 331-40; Wiebalck A et al, “Sind Tramadol-Enantiomere für die postoperative Schmerztherapie besser geeignet als das Racemat? Eine randomisierte, Plazebo- and Morphin-kontrollierte Doppelblindstudie”, Der Anaesthesist, 1998; 47: 387-394). It is obvious to conclude that the potent analgesic action of tramadol is based on this mutually dependent reinforcement of action of the enantiomers. Tramadol's major active metabolite, 0-desmethyltramadol (M1), shows higher affinity for the μ-opiate receptor and has at least twice the analgesic potency of the parent drug. O-desmethyl-N-mono-desmethyltramadol (referred to as M5 in some places in the following text and in the literature) is known as one of the in vivo metabolites of tramadol (1RS, 2R5)-2[(dimethylamino)methyl]-1-(3-methoxyphenyl)cyclohexanol (Lintz et al, Arzneim.-Forsch./Drug Res. 1981; 31(11): 1932-1943). M5 penetrates the blood-brain barrier to only a limited extent, as the effects on the central nervous system, for example analgesic effects, are distinctly less pronounced on intravenous administration than on intracerebroventricular administration. Despite the fact that tramadol is chemically unrelated to the opiates adverse side effects associated with administration of tramadol are similar to those of the opiates.

Unlugenc et al, (A comparative study on the analgesic effect of tramadol, tramadol plus magnesium, and tramadol plus ketamine for postoperative pain management after major abdominal surgery. Acta anaesthesiologica Scandinavica 2002; 46:1025-30) have shown that adding magnesium or ketamine to tramadol improved analgesia and patient comfort and decreased the amount of tramadol required for postoperative pain management after major abdominal surgery. Chen et al, (Isobolographic analysis of the analgesic interactions between ketamine and tramadol. Journal of pharmacy and pharmacology 2002; 54:623-31) have shown that in the acute thermal or chemical pain model, ketamine is not effective and the net effect of ketamine and tramadol in combination was simply additive after systemic administration. However, the co-administration produced synergistic antinociception in the chemical-induced persistent pain model.

In summary, tramadol and its active metabolite M1, modulate neuronal pathways via contributions from both opioid (predominantly at the μ-opioid receptor) and non-opioid probably related to its inhibition of neuronal release or reuptake of norepinephrine and serotonin) mechanisms at therapeutic doses. Both mechanisms contribute to the effect of tramadol in vivo, leading to the suggestion that tramadol is a novel centrally acting analgesic that mimics, in a single drug substance, the clinical practice of combining opioid analgesics with monoamine reuptake inhibitors. Opioid receptors presynaptically inhibit transmission of excitatory pathways. These pathways include acetylcholine, the catecholamines, serotonin, and substance P. The present working hypothesis is that the overall neuronal action of tramadol is dependent on the different pharmacologies of its enantiomers and, to some extent its metabolite, M1. The enantiomers appear to interact in a complementary and synergistic manner to produce antinociception, but only in an additive or counteractive manner on adverse-effect end-points. Hence, the favorable clinical profile of tramadol appears to be a consequence of the fortuitous interaction of the enantiomers and the metabolite M1 on the therapeutic endpoint, but not on adverse-effect endpoints.

### 10). Alpha-2-Delta Ligands

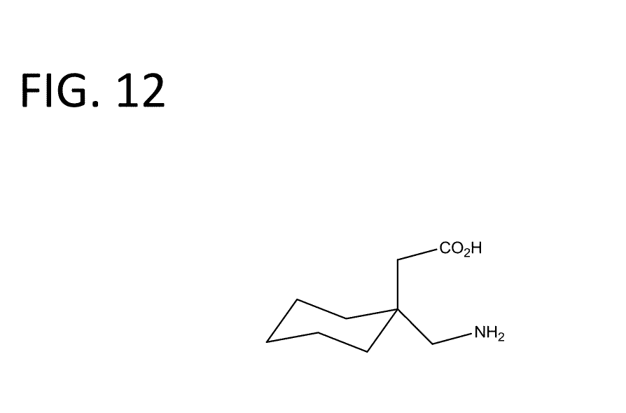

Gabapentin (GBP; Neurontin; FIG. 12) is an anticonvulsant that has found increased utility for the treatment of clinical neuropathic pain. Although originally developed for the treatment of spasticity and epilepsy, recent attention has focused on the utility of GBP for the treatment of neuropathic pain based on its efficacy and minimal side-effect profile in clinical trials (Rice A S C and Maton S (2001) Gabapentin in postherpetic neuralgia: a randomised, double blind, placebo controlled study. Pain 94: 215-224).

Gabapentin and pregabalin are known to interact with both the α₂δ-1 and α₂δ-2 subunits (Klugbauer, N, Marais, E & Hofmann, F. (2003) J Bioenerg Biomembr 35, 639-647). The specific binding of gabapentin to α₂δ-1 was the first described interaction between a regulatory subunit of voltage activated calcium channels and a pharmaceutical agent. The discovery of the α₂δ subunit of voltage-gated calcium channels as a high-affinity binding site for GBP has further supported a role for voltage-gated calcium channels in its antinociceptive action.

It has been shown that α2δ-1 is up-regulated in DRG neurons after nerve injury (Luo, Z D, et al., (2002) Pharmacol Exp Ther 303, 1199-1205) and that this correlates with the onset and duration of pain behavior. Previous pharmacological investigations have provided evidence that analgesic activity of pregabalin and gabapentin is through the α2δ subunit. Recent results are consistent with and add to these findings, demonstrating that it is the α₂δ-1 subunit that provides the requirement for the analgesic action of pregabalin and gabapentin (Field M J et al. (2006) Identification of the α2δ-1 subunit of voltage-dependent calcium channels as a molecular target for pain mediating the analgesic actions of pregabalin; PNAS; 103: 17537-17542).

In summary, Gabapentin interacts with both the α₂δ-1 and α₂δ-2 subunits which are voltage-gated calcium channel thus blocking calcium influx into the neuronal cells. A specific role for α₂δ in neuropathic pain is due to the fact that an increase in α₂δ expression in the dorsal root ganglion ipsilateral to the peripheral nerve injury that corresponded to the development of tactile allodynia. In addition, gabapentin increases brain extracellular GABA levels in both rat and human studies which is partially responsible for its effectiveness for neuropathic pain, since the pathology associated with this condition includes disruption of tonic inhibitory GABAergic transmission.

### 11). Aloe Vera Gel and Juice

Aloe vera's use can be traced back 6,000 years to early Egypt, where the plant was depicted on stone carvings. Known as the “plant of immortality,” aloe was presented as a burial gift to deceased pharaohs. Traditionally, aloe was used topically to heal wounds, inflammation, and for various skin conditions, and orally as a laxative. In addition to traditional uses, people take aloe orally to treat a variety of conditions, including diabetes, asthma, epilepsy, and osteoarthritis. People use aloe topically for osteoarthritis, burns, sunburns, and psoriasis. Aloe leaves contain a clear gel that is often used as a topical ointment. Aloe vera gel can be found in hundreds of skin products, including lotions and sunblocks.

### 12). Santalol, Santalol Acetate, Amyris Alcohol and Amyris Acetate

The composition of the present invention can contain one or more agents selected from santalol, santalyl acetate, amyris alcohol and amyris acetate to improve the efficacy in treating fever blisters and cold sores.

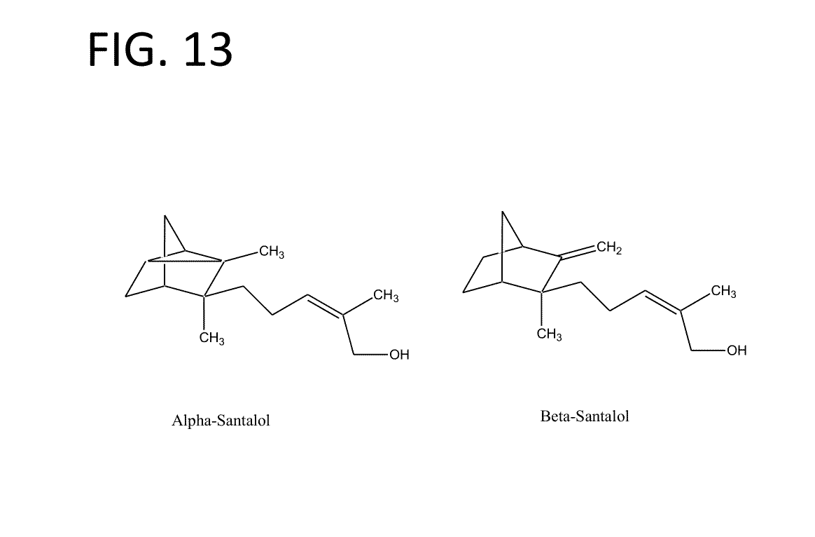

The main constituent of sandalwood oil is santalol (FIG. 13). This primary sesquiterpene alcohol forms more than 90 per cent of the oil and is present as a mixture of two isomers, α-santalol and β-santalol, the former predominating. The inventors have disclosed in the U.S. Pat. No. 7,858,126, the use of esters of santalol as anti-bacterial and anti-viral agents in mammals in vivo. Specifically, the santalyl acetate has been shown to be useful for treating fever blisters and cold sores.

Amyris oil which is obtained from amyris tree (Amyris balsamifera) is rich in sesquiterpene alcohols (60-85%), e.g. valerianol, eudesmol (α, β and γ isomers) and elemol (Van T B A, Kleis R, et al. (1989). Essential oil of Amyris balsamifera. Phytochemistry 28(7): 1909-1912). The oil is further refined under vacuum to produce amyris alcohol which is a mixture of sesquiterpene alcohols including valerianol, beta-eudesmol, epi-gamma-eudesmol, elemol and alpha-eudesmol. Amyris acetate is obtained by fully acetylating amyris alcohol and is a mixture of esters of sesquiterpene alcohols.

U.S. Patent Application Publication No. 2010/0120907 relates to topical formulations comprising amyris alcohol and/or ester derivatives of amyris alcohol which may be used for the treatment of diseases including herpes virus infection (e.g., HSV-1, HSV-2), epidermoid carcinoma, cold sores, and human papillomavirus.

### D. SKIN PERMEATION ENHANCERS

The pharmaceutical compositions of the present invention can contain one or more skin permeation enhancers. The skin permeation enhancers which are suitable for incorporating into the compositions of the present inventions include but not limited to fatty alcohols such as decanol, lauryl alcohol, linolenyl alcohol, n-octanol and oleyl alcohol; fatty acid esters such as ethyl acetate, dodecyl N,N-dimethylamino acetate, glycerol monolaurate, glycerol monooleate, isopropyl myristate, methyl laurate and sorbitan monooleate; fatty acids such as lauric acid and oleic acid; biologics such as lecithin, amines and amides such as N,N-dimethyl-m-toluamide, lauryl-amine and urea; complexing agents such as cyclodextrin, hydroxypropyl methylcellulose and liposomes; surfactants such as Brij 36T, sodium lauryl sulfate and sorbitan monooleate; other compounds such as dimethyl isosorbide, bisabolol, eucalyptol, menthol, terpenes, N-methyl pyrrolidone, azone, DMSO, MSM, decylmethyl sulfoxide, dimethyl formamide, dimethyl acetamide, glycols and propylene glycol.

### E. PHARMACEUTICAL COMPOSITIONS

In some embodiments, the invention provides pharmaceutical compositions comprising the esters of capsaicins in high concentration set forth herein.

The phrases “pharmaceutical,” “pharmaceutically,” or “pharmacologically acceptable” refer to molecular entities and compositions that do not produce an unacceptably adverse, allergic or other untoward reaction when administered to an animal, or human, as appropriate. As used herein, “pharmaceutical” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients to treat the disease of interest, such as other anti-cancer agents can also be incorporated into the compositions.

Pharmaceutical compositions of the present invention comprise one or more of the ester derivatives of capsaicin set forth herein. When used for therapy, the compositions of the present invention are administered to subjects in therapeutically effective amounts. For example, an effective amount of the ester of capsaicin in a patient with diabetic neuropathy may be an amount that promotes the healing of the pain associated with the neuropathy. The dose will depend on the nature of the disease, the subject, the subject's history, and other factors. Preparation of such compositions is discussed in other parts of this specification.

As discussed above, the derivatives of capsaicin set forth herein have greater lipophilicity and significantly non-burning to the skin even at extremely high concentration as compared to capsaicin. Another advantage is that these compositions have a very low toxicity to the skin as compared to formulations of capsaicin.

The compositions of the capsaicin derivatives of the present invention can be delivered by any method known to those of ordinary skill in the art. For example, the pharmaceutical compositions can be delivered by topical delivery routes.

Compositions employing the esters of capsaicin set forth herein can contain high concentration or in high concentration of the derivative. As used herein a high concentration of a compound or composition refers to an amount higher than at least 10% by weight. For example, in one embodiment, the invention provides a composition comprising capsaicin palmitate ester present in an amount of about 14.25% by weight, which corresponds to about 8% of capsaicin by weight.

In some embodiments, the invention provides pharmaceutical compositions comprising esters of capsaicin present in amounts ranging from about 10% to about 50% by weight, from about 10% to about 40% by weight, from about 10% to about 30% by weight, from about 10 to about 25% by weight, from about 10% to about 20% by weight and from about 10% to about 15% by weight. In some embodiments, the invention provides pharmaceutical compositions comprising esters of capsaicin in amounts of about 14.25% by weight. In some embodiments, the pharmaceutical composition is formulated for topical administration.

In some embodiments, the pharmaceutical compositions of the present invention can additionally comprise one or more other pain relieving agents. In some embodiments, the pharmaceutical composition further comprises a pain relieving agent selected from the group consisting of salicylates, menthol, boswellic acids, DMSO, methyl sulfonylmethane, NSAIDs, corticosteroids, emu oil, opioid agonists and antagonists, NMDA antagonists, tramadol, hyaluronic acid, α2δ ligands, aloe vera gel and aloe vera juice for improved pain relieving properties. The type and amount of other pain relieving agents which can be incorporated into the pharmaceutical compositions set forth herein depends on the information that are clinically determined to be useful in the treatment of the particular disease under consideration. One of ordinary skill in the art would be familiar with what type of dosage is required for treatment of the particular pathological condition that is present in the subject. No undue experimentation would be involved. When used for therapy, the compositions of the present invention which contain other pain relieving agents are administered to subjects in therapeutically effective amounts. For example, an effective amount of other pain relieving agent which can be combined with the esters of capsaicin to treat a patient with diabetic neuropathy may be an amount that promotes the healing of the pain associated with the neuropathy. The dose will depend on the nature of the disease, the subject, the subject's history, and other factors. Preparation of such compositions is discussed in other parts of this specification.

The pain relieving agents listed in the above paragraph can also be combined and administered with the esters of capsaicin of the present invention in separate compositions. In some embodiments, the separate compositions are administered simultaneously while in other embodiments, the separate compositions are not administered simultaneously, such as, for example, in a predetermined sequential manner.

In some embodiments, additional therapeutic agents can be combined in the same composition as the composition comprising the esters of capsaicin and/or the one or more other pain relieving agents. In some embodiments, the additional therapeutic agents can be administered in separate compositions. In some embodiments, the additional therapeutic agents can be selected from santalol, santalyl acetate, amyris alcohol and amyris acetate to improve the efficacy in treating fever blisters and cold sores.

The therapeutic agents of the present invention may be supplied in any form known to those of ordinary skill in the art. For example, the therapeutic agent may be supplied as a liquid or as a solution. In some embodiments, the pharmaceutical compositions may contain a preservative to prevent the growth of microorganisms. In some embodiments, the pharmaceutical compositions are chemically and physically stable under the conditions of manufacture and storage and must be preserved against any contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like.

The formulations according to the invention having been described herein may influence the ordinarily skilled artesian to make similar formulations using components that will be known in the art, without departing from the invention which is claimed herein.

The pharmaceutical formulations of the esters of capsaicin according to the present invention offer several advantages over the existing formulations. They can be topically applied and relatively high concentrations of the esters of capsaicin can be loaded into patients with high bioavailability. Thus the frequency of dosage can be reduced. Thus within the spirit, the invention is related to improved formulations and methods of using the same when administering such formulations to patients. As mentioned herein above a number of excipients may be appropriate for use in the formulation which comprises the composition according to the present invention. The inclusion of excipients and the optimization of their concentration for their characteristics such as for example ease of handling or carrier agents will be understood by those ordinarily skilled in the art not to depart from the spirit of the invention as described herein and claimed herein below.

Following preparation of the pharmaceutical compositions of the present invention, it may be desirable to quantify the amount of the esters of capsaicin in the pharmaceutical composition. Methods of measuring concentration of a drug in a composition include numerous techniques that are well-known to those of skill in the art. Selected examples include chromatographic techniques. There are many kinds of chromatography which may be used in the present invention: drug-specific assays, adsorption, partition, ion-exchange and molecular sieve, and many specialized techniques for using them including column, paper, thin-layer chromatography, gas chromatography, and high performance liquid chromatography (HPLC). One of ordinary skill in the art would be familiar with these and other related techniques.

In order to verify the non-burning aspect the composition of the present invention, a 14.25% of capsaicin palmitate ointment was prepared as in Example 2. Additionally, an 8% capsaicin ointment was also prepared in the same manner. About 1 gram of the capsaicin palmitate ointment was applied at one of the forehand of six healthy volunteers and about 1 gram of the 8% capsaicin ointment was applied at the other forehand of the volunteers. After 30 minutes, the volunteers were asked about the burning sensation at both arms. The capsaicin palmitate did not cause any burning sensation at the site of application while the capsaicin caused intense burning at the site of application and the pain lasted for almost 48 hours.

### F. MOISTURIZING AGENTS

In some embodiments, certain topical formulations of the present invention may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

### G. ANTIOXIDANTS

In some embodiments, certain topical formulations of the present invention may contain one or more antioxidants. Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

### H. PATHOLOGICAL CONDITIONS TO BE TREATED OR PREVENTED

The term “treat” or “treatment” means that the symptoms associated with one or more conditions mentioned above are alleviated or reduced in severity or frequency and the term “prevent” means that subsequent occurrences of such symptoms are avoided or that the frequency between such occurrences is prolonged.

Examples of pathological conditions responsive to capsaicin therapy include, but are not limited to, post-herpetic neuralgia, shingles (herpes zoster), diabetic neuropathy, postmastectomy pain syndrome, oral neuropathic pain, trigeminal neuralgia, temperomandibular joint disorders, pruritus, cluster headache, osteoarthritis, arthritis pain, rhinopathy, oral mucositis, cutaneous allergy, detrusor hyperreflexia, loin pain/hematuria syndrome, neck pain, amputation stump pain, reflex sympathetic dystrophy and pain due to skin tumor.

The pharmaceutical compositions comprising ester derivatives of capsaicin set forth herein are useful in the treatment and prevention of any of the diseases set forth above. One of ordinary skill in the art would be familiar with the many diseases and conditions that would be amenable to treatment with one or more of the ester derivatives of capsaicin set forth herein.

### H. SECONDARY THERAPIES

Some embodiments of the claimed methods of the present invention involve administering to the subject a secondary form of therapy in addition to one or more of the therapeutic combination of ester derivatives of capsaicin set forth herein. For example, if the disease is a hyperproliferative disease, such as cancer, the secondary therapy may be a chemotherapeutic agent, radiation therapy, surgical therapy, immunotherapy, gene therapy, or other form of anticancer therapy well-known to those of ordinary skill in the art. If the disease is an inflammatory disease such as arthritis, exemplary secondary forms of therapy include non-steroidal anti-inflammatory agents, steroids and immunosuppressant therapy.

In order to increase the effectiveness of the therapeutic agent disclosed herein, it may be desirable to combine the therapeutic agent of the present invention with the secondary therapeutic agent. These compositions would be provided in a combined amount effective to provide for a therapeutic response in a subject. One of ordinary skill in the art would be able to determine whether the subject demonstrated a therapeutic response. This process may involve administering the therapeutic agent of the present invention and the secondary therapeutic agent to the subject at the same time. This may be achieved by administering a single composition or pharmacological formulation that includes both agents, or by administering two distinct compositions or formulations, at the same time, wherein one composition includes the ester derivative of capsaicin of the present invention and the other includes the secondary agent.

Alternatively, the therapeutic agent of the present invention may precede or follow the treatment with the secondary agent by intervals ranging from minutes to weeks. In embodiments where the secondary agent and the ester derivatives of the present invention are separately administered, one would generally ensure that a significant period of time did not expire between the time of each delivery, such that the secondary agent and the therapeutic agent of the present invention would still be able to exert a beneficial effect on the subject. In such instances, it is contemplated that one may administer both modalities within about 24-48 h of each other and, more preferably, within about 12-24 h of each other, and even more preferably within about 30 minute-6 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the therapeutic agent of the present invention is “A” and the secondary agent, such as chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient will follow general protocols for the administration of therapeutic agents, such as chemotherapy where the disease to be treated is cancer. It is expected that the treatment cycles would be repeated as necessary.

### I. EXAMPLES

The following examples are included to demonstrate some embodiments of the invention. It should be appreciated by those of skill in the art that the techniques disclosed in the examples which follow represent techniques discovered by the inventor to function in the practice of the invention, and thus can be considered to constitute operative modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Palmitoyl Ester of Capsaicin USP (Formula I, R=—(CH₂)₁₄—CH₃)

A mixture of 1478 μm of capsaicin USP (HUBEI XIANGXI CHEMICAL INDUSTRY CO., LTD, China), 700 ml of anhydrous triethylamine (Spectrum Chemicals) and 7400 ml of anhydrous dichloromethane was charged into a 50 L double jacketed chemical glass reactor. The content was covered with aluminum foil to protect it from light exposure. The reactor was fitted with a condenser fitted with a moisture trap on the top and a drop wise addition funnel. The flask was kept at room temperature and 1260 ml of palmitoyl chloride was added from the funnel into the mixture slowly with stirring. After the addition, the mixture was refluxed for 8 hours and stirred for 10-15 hours at 40° C. temperature. The mixture was washed successively with 2×20 L of water, 2×20 L of 0.1N dilute hydrochloric acid, 2×20 L of 10% sodium bicarbonate solution and 3×20 L of type I water until the washed out solution was neutral. The organic layer was separated, dried with anhydrous magnesium sulfate and the dichloromethane was removed under vacuum to produce a clear, yellow waxy solid (95% of theoretical).

The capsaicin palmitate was thoroughly dried and then weighed, (W_{CP}), in grams. This weight was multiplied by 1.21 to obtain the total volume when the capsaicin palmitate is dissolved in methanol, and this quantity was noted as the dissolved volume (D_{V}). The amount of methanol, X, in milliliters, to be added to make a 4% solution was calculated by the equation below:

$$X = \frac{{W}_{CP}}{0.04} - {D}_{V} \qquad ( II )$$

The weighed capsaicin palmitate was transferred into a suitable container and the calculated amount of methanol was added to it. The solution was stirred while applying low heat to the container in a water bath; the solids was completely dissolved either under mild to high shear stirring between 25° C. to 35° C., or under low shear stirring between 35° C. to 45° C.

The solution was filtered through a 11 μm or smaller filter media to remove any dust or insoluble particles and this clean solution was transferred into a crystallization vessel (such as a cylinder) and then the top was covered with laboratory film and aluminum foil. The vessel was refrigerated at 4° C. and kept it there for 8-12 hours, or until no more precipitate formed. The precipitate was removed and the supernatant was transferred to a suitably sized Buchner funnel fitted with Grade 2 or 3 qualitative filter paper. The filtered solution was collected under vacuum and set aside. The filter cake was washed with methanol that has been cooled to 4° C., using approximately 1 L of methanol for every 250 g of capsaicin palmitate that was initially dissolved. These washed solution was collected under vacuum and combined them with the solution that was collected earlier and set aside. The filter cake was dried over vacuum until no more solution was coming over into the collection flask. The cake was transferred and evenly divided in pyrex glass trays and then placed the trays into an vacuum oven. The capsaicin palmitate was dried under high vacuum at 35° C. for 12 hours, or until there is no more methanol odor and the material is dry and crumbled to the touch.

The recrystallized capsaicin palmitate was weighed, recording the weight (W_{obtained}) in grams. The remaining capsaicin palmitate in the filtrate solution (W_{remaining}) was calculated as,

W_{CP}−W_{obtained}=W_{remaining}.

The excess solvent was removed from the filtrate using a rotary evaporator so that the volume of the solvent left would satisfy the equation II. The concentrated solution was refrigerated at 4° C. as described in the previous paragraph to obtain the crystallized capsaicin plamitate and processed as described in the previous paragraph. The crystallized capsaicin palmitate from the two steps was combined together and the yield was calculated.

The crystallization was repeated one more time to obtain capsaicin palmitate with less than 0.005% of capsaicin as impurity.

### Example 2

### Preparation of 14.25% Capsaicin Palmitate Ointment and Liquid

### Formulation 1. Ointment

The following ingredients were weighed accurately and mixed in a 100 mL beaker while heating at 70° C. The mixture was cooled to room temperature and mixed again to obtain the specified ointment.

Capsaicin Palmitate (14.25%) Composition Ointment

| | | | | Jar (gm) 5 |
|---|---|---|---|---|
| Batch Size (gm) 50.0 | | CP Overage (%) 5 | | Amount |
| Name of Ingredient | | Percent | | Batch (gm) |
| 1 | Cetyl Myristoleate | 10.000 | | 5.000 |
| 2 | Oleyl Alcohol | 20.000 | | 10.000 |
| 3 | Capsaicin Palmitate | 14.250 | 14.963 | 7.481 |
| 4 | Isopropyl Myristate | 20.000 | | 10.000 |
| 5 | Lavender Oil | 2.000 | | 1.000 |
| 6 | Glyceryl Monooleate | 10.000 | | 5.000 |
| 7 | PEG 400 | 4.000 | | 2.000 |
| 8 | Polysorbate 80 | 3.000 | | 1.500 |
| 9 | Propylene Glycol | 5.000 | | 2.500 |
| 10 | Vitamin E Acetate | 2.000 | | 1.000 |
| 11 | White Petroleum | 5.000 | | 2.500 |
| 12 | Cetearyl Alcohol | 4.750 | 4.038 | 2.019 |
| | TOTAL | 100.000 | | 50.000 |

Total # of Jars 10

### Formulation 2. Liquid

The following ingredients were weighed accurately and mixed in a 100 mL beaker while heating at 70° C. The mixture was cooled to room temperature and mixed again to obtain the specified liquid.

Capsaicin Palmitate (14.25%) Liquid Composition

| | | | | Bottle (gm) 10 |
|---|---|---|---|---|
| Batch Size (gm) 50.0 | | CP Overage (%) 5 | | Amount |
| Name of Ingredient | | Percent | | Batch (gm) |
| 1 | Cetyl Myristoleate | 15.000 | | 7.500 |
| 2 | Eugenyl Acetate | 1.000 | | 0.500 |
| 3 | Capsaicin Palmitate | 14.250 | 14.963 | 7.481 |
| 4 | Lavender Oil | 2.000 | | 1.000 |
| 5 | Isopropyl Myristate | 10.000 | | 5.000 |
| 6 | Vitamin E Acetate | 2.000 | | 0.644 |
| 7 | Glyceryl Monooleate | 55.750 | 55.038 | 27.875 |
| | TOTAL | 100.000 | | 50.000 |

Total # of Bottle 5

### Example 3

### Preparation of Capsaicin Palmitate (0.445%) and Menthol (3%) Cream

The ingredients listed in the following Table were separated into oil phase and water phase ingredients except benzyl alcohol. The oil phase ingredients were weighed accurately and transferred to a 500 mL beaker and heated to 60-70° C. The water phase ingredients were weighed accurately and transferred to a 1 L glass bowl and heated to 60-70° C. while stirring to form a homogeneous solution. The water phase was cooled to the room temperature and the oil was added slowly to the water phase with rapid mixing. Benzyl alcohol was added to the cream while rapidly mixing. The resultant cream was mixed for 10 minutes and allowed to cool to the room temperature.

Menthol (3%)& Capsaicin Palmitate (0.445%) Cream

| | | | | Jar (gm) 35 |
|---|---|---|---|---|
| Batch Size (Gm) 500.0 | | CP Overage (%) 5 | | Amount |
| Name of Ingredient | | Percent | | Batch (gm) |
| 1 | Menthol | 3.000 | | 15.00 |
| 2 | Camphor | 2.000 | | 10.00 |
| 2 | Capsaicin Palmitate | 0.445 | 0.467 | 2.34 |
| 3 | Lavender Oil | 0.000 | | 0.00 |
| 4 | Hallbrite BHB | 6.000 | | 30.00 |
| 5 | Glyceryl Monooleate | 5.000 | | 25.00 |
| 6 | Vitamin E Acetate | 2.000 | | 10.00 |
| 7 | Cetearyl Alcohol | 5.000 | | 25.00 |
| 7 | Cetyl Palmitate | 8.000 | | 40.00 |
| 9 | PEG 400 | 4.000 | | 20.00 |
| 10 | Polysorbate 80 | 3.000 | | 15.00 |
| 11 | Glycerin | 8.000 | | 40.00 |
| 12 | Methyl Paraben | 0.100 | | 0.50 |
| 13 | Propyl Paraben | 0.010 | | 0.05 |
| 14 | Xanthn Gum | 0.500 | | 2.50 |
| 15 | Benzyl Alcohol | 2.000 | | 10.00 |
| | Water | 50.945 | 50.923 | 254.61 |
| | TOTAL | 100.000 | | 500.00 |

Total # of Jars 14

### Example 4

### Preparation of Capsaicin Palmitate (0.445%) and Methyl Salicylate (10%) Cream

The ingredients listed in the following Table were separated into oil phase and water phase ingredients except benzyl alcohol. The cream was prepared as described in Example 3.
In a similar manner, the cream composition containing capsaicin palmitate (0.445%) and methyl acetylsalicylate (10%) was prepared.

Methyl Salicylate (10%) + Capsaicin
Palmitate (0.445%) Composition Cream

| | | | | Jar (gm) 35 |
|---|---|---|---|---|
| Batch Size (gm) 500.0 | | CP Overage (%) 8 | | Amount |
| Name of Ingredient | | Percent | | Batch (gm) |
| 1 | Methyl Salicylate | 10.000 | | 50.000 |
| 2 | Capsaicin Palmitate | 0.445 | 0.481 | 2.403 |
| 3 | Lavender Oil | 2.000 | | 10.000 |
| 4 | Hallbrite BHB | 5.000 | | 25.000 |
| 5 | Glyceryl Monooleate | 3.500 | | 17.500 |
| 6 | Vitamin E Acetate | 2.000 | | 10.000 |
| 7 | Cetearyl Alcohol | 6.000 | | 30.000 |
| 7 | Cetyl Palmitate | 5.000 | | 25.000 |
| 9 | PEG 400 | 4.000 | | 20.000 |
| 10 | Polysorbate 80 | 3.000 | | 15.000 |
| 11 | Glycerin | 7.000 | | 35.000 |
| 12 | Methyl Paraben | 0.100 | | 0.500 |
| 13 | Propyl Paraben | 0.010 | | 0.050 |
| 14 | Xanthn Gum | 0.500 | | 2.500 |
| 15 | Benzyl Alcohol | 2.000 | | 10.000 |
| | Water | 49.445 | 49.409 | 247.05 |
| | TOTAL | 100.000 | | 500.000 |

Total # of Jars 14

### Example 5

### Preparation of Capsaicin Palmitate (0.445%) and EMU OIL (10%) Cream

The ingredients listed in the following Table were separated into oil phase and water phase ingredients except benzyl alcohol. The cream was prepared as described in Example 3.

EMU OIL (10%) + Capsaicin
Palmitate (0.445%) Composition Cream

| | | | | Jar (gm) 35 |
|---|---|---|---|---|
| Batch Size (Gm) 500.0 | | CP Overage (%) 8 | | Amount |
| Name of Ingredient | | Percent | | Batch (gm) |
| 1 | EMU OIL | 10.000 | | 50.000 |
| 2 | Capsaicin Palmitate | 0.445 | 0.481 | 2.403 |
| 3 | Lavender Oil | 2.000 | | 10.000 |
| 4 | Hallbrite BHB | 5.000 | | 25.000 |
| 5 | Glyceryl Monooleate | 3.500 | | 17.500 |
| 6 | Vitamin E Palmitate | 2.000 | | 10.000 |
| 7 | Cetearyl Alcohol | 6.000 | | 30.000 |
| 7 | Cetyl Palmitate | 5.000 | | 25.000 |
| 9 | PEG 400 | 4.000 | | 20.000 |
| 10 | Polysorbate 80 | 3.000 | | 15.000 |
| 11 | Glycerin | 7.000 | | 35.000 |
| 12 | Methyl Paraben | 0.100 | | 0.500 |
| 13 | Propyl Paraben | 0.010 | | 0.050 |
| 14 | Xanthn Gum | 0.500 | | 2.500 |
| 15 | Benzyl Alcohol | 2.000 | | 10.000 |
| | Water | 49.445 | 49.409 | 247.05 |
| | TOTAL | 100.000 | | 500.000 |

Total # of Jars 14

### Example 6

### Preparation of Capsaicin Palm Itate (0.445%) and Ibuprofen (5%) Cream

The ingredients listed in the following Table were separated into oil phase and water phase ingredients except benzyl alcohol. The cream was prepared as described in Example 3.

IBUPROFEN (5%) + Capsaicin
Palmitate (0.445%) Composition Cream

| | | | | Jar (gm) 35 |
|---|---|---|---|---|
| Batch Size (Gm) 500.0 | | CP Overage (%) 8 | | Amount |
| Name of Ingredient | | Percent | | Batch (gm) |
| 1 | IBUPROFEN | 5.000 | | 25.000 |
| 2 | Capsaicin Palmitate | 0.445 | 0.481 | 2.403 |
| 3 | Lavender Oil | 2.000 | | 10.000 |
| 4 | Hallbrite BHB | 7.000 | | 35.000 |
| 5 | Glyceryl Monooleate | 3.500 | | 17.500 |
| 6 | Vitamin E Acetate | 2.000 | | 10.000 |
| 7 | Cetearyl Alcohol | 6.000 | | 30.000 |
| 7 | Cetyl Palmitate | 5.000 | | 25.000 |
| 9 | PEG 400 | 4.000 | | 20.000 |
| 10 | Polysorbate 80 | 3.000 | | 15.000 |
| 11 | Propylene Glycol | 10.000 | | 50.000 |
| 12 | Methyl Paraben | 0.100 | | 0.500 |
| 13 | Propyl Paraben | 0.010 | | 0.050 |
| 14 | Xanthn Gum | 0.500 | | 2.500 |
| 15 | Benzyl Alcohol | 2.000 | | 10.000 |
| | Water | 49.445 | 49.409 | 247.05 |
| | TOTAL | 100.000 | | 500.000 |

Total # of Jars 14

### Example 7

### Preparation of Capsaicin Palmitate (0.445%) and MSM (10%) Cream

The ingredients listed in the following Table were separated into oil phase and water phase ingredients except benzyl alcohol. The cream was prepared as described in Example 3.
In a similar manner, the cream composition containing capsaicin palmitate (0.445%) and DMSO (10%) was prepared.

MSM (10%) + Capsaicin Palmitate (0.445%) Composition Cream

| | | | | Jar (gm) 35 |
|---|---|---|---|---|
| Batch Size (gm) 500.0 | | CP Overage (%) 8 | | Amount |
| Name of Ingredient | | Percent | | Batch (gm) |
| 1 | MSM | 10.000 | | 50.000 |
| 2 | Capsaicin Palmitate | 0.445 | 0.481 | 2.403 |
| 3 | Lavender Oil | 2.000 | | 10.000 |
| 4 | Hallbrite BHB | 7.000 | | 35.000 |
| 5 | Glyceryl Monooleate | 3.500 | | 17.500 |
| 6 | Vitamin E Acetate | 2.000 | | 10.000 |
| 7 | Cetearyl Alcohol | 6.000 | | 30.000 |
| 7 | Cetyl Palmitate | 5.000 | | 25.000 |
| 9 | PEG 400 | 4.000 | | 20.000 |
| 10 | Polysorbate 80 | 3.000 | | 15.000 |
| 11 | Propylene Glycol | 5.000 | | 25.000 |
| 12 | Methyl Paraben | 0.100 | | 0.500 |
| 13 | Propyl Paraben | 0.010 | | 0.050 |
| 14 | Xanthn Gum | 0.500 | | 2.500 |
| 15 | Benzyl Alcohol | 2.000 | | 10.000 |
| | Water | 49.445 | 49.409 | 247.05 |
| | TOTAL | 100.000 | | 500.000 |

Total # of Jars 14

### Example 8

### Preparation of Prednisolone Acetate (0.5%) and Capsaicin Palmitate (0.445%) Ointment

The ingredients listed in the following Table were weighed accurately and transferred to a 100 mL beaker and the content was heated to 70 C while stirring. The homogeneous mixture was allowed to cool to room temperature under constant stirring to produce the ointment.
In a similar manner, the ointment composition containing capsaicin palmitate (0.445%) and Hydrocortizone (0.5%) was prepared.

Prednisolone Acetate (0.5%) and Capsaicin
Palmitate (0.445%) Composition Ointment

| | | | | Jar (gm) 5 |
|---|---|---|---|---|
| Batch Size (GM) 50.0 | | CP and PA Overage (%) 5 | | Amount |
| Name of Ingredient | | Percent | | Batch (GM) |
| 1 | Prednisolone Acetate | 0.500 | 0.525 | 0.263 |
| 2 | Oleyl Alcohol | 3.000 | | 1.500 |
| 3 | Capsaicin Palmitate | 0.445 | 0.467 | 0.234 |
| 4 | Isopropyl Myristate | 3.000 | | 1.500 |
| 5 | Lavender Oil | 2.000 | | 1.000 |
| 6 | Glyceryl Monooleate | 4.000 | | 2.000 |
| 7 | PEG 400 | 4.000 | | 2.000 |
| 8 | Polysorbate 80 | 3.000 | | 1.500 |
| 9 | Cetearyl Alcohol | 5.000 | | 2.500 |
| 10 | Vitamin E Acetate | 1.000 | | 0.500 |
| 11 | White Petroleum | 5.000 | | 2.500 |
| 12 | Propylene Glycol | 69.055 | 69.008 | 34.504 |
| | TOTAL | 100.000 | | 50.000 |

Total # of Jars 10

### Example 9

### Capsule Formulation Containing Capsaicin Palmitate, Gabapentin and Tramadol

The following ingredients required for 1000 capsules were weighed accurately, mixed using a high shear mixer to fine and homogeneous powder. The powder was sieved through 100 mesh and filled into hard gelatin capsules. The composition of each capsule formulation is listed below.

### Capsule Formulation 1.

Capsule Formulation Capsaicin Palmitate +
Tramadol + Gabapentin

| | | | Number of Capsules | | |
|---|---|---|---|---|---|
| | | | 100 | 1,000 | |
| | | TLI-0306 | | Overage (%) 5 | |
| | NAME OF INGREDIENT | mg | gm | gm | % |
| 1 | Capsaicin Palmitate | 5.40 | 0.567 | 5.67 | 1.69 |
| 2 | Tramadol•HCl | 50.00 | 5.250 | 52.50 | 15.63 |
| 3 | Gabapentin | 100.00 | 10.500 | 105.00 | 31.25 |
| 5 | Microcrystalline Cellulose | 58.40 | 5.063 | 50.63 | 18.25 |
| 6 | Silicon Dixode | 3.00 | 0.300 | 3.00 | 0.94 |
| 7 | Sodium Lauryl Sulfate | 1.60 | 0.160 | 1.60 | 0.50 |
| 8 | Magnesium Stearate | 1.60 | 0.160 | 1.60 | 0.50 |
| 9 | MgSO4 | 100.00 | 10.000 | 100.00 | 31.25 |
| | Total | 320.00 | 32.000 | 320.00 | 100.00 |
| | Capsule Size | 1 | 1 | 1 | |

No of capsules per bottle 30
No of bottles 33

### Capsule Formulation 2.

Capsule Formulation Capsaicin Palmitate +
Tramadol + Gabapentin

| | | | Number of Capsules | | |
|---|---|---|---|---|---|
| | | | 100 | 1,000 | |
| | | TLI-0306 | | Overage (%) 5 | |
| NAME OF INGREDIENT | | mg | gm | gm | % |
| 1 | Capsaicin Palmitate | 10.80 | 1.134 | 11.34 | 3.38 |
| 2 | Tramadol•HCl | 50.00 | 5.250 | 52.50 | 15.63 |
| 3 | Gabapentin | 100.00 | 10.500 | 105.00 | 31.25 |
| 5 | Microcrystalline Cellulose | 53.00 | 4.496 | 44.96 | 16.56 |
| 6 | Silicon Dixode | 3.00 | 0.300 | 3.00 | 0.94 |
| 7 | Sodium Lauryl Sulfate | 1.60 | 0.160 | 1.60 | 0.50 |
| 8 | Magnesium Stearate | 1.60 | 0.160 | 1.60 | 0.50 |
| 9 | MgSO4 | 100.00 | 10.000 | 100.00 | 31.25 |
| | Total | 320.00 | 32.000 | 320.00 | 100.00 |
| | Capsule Size | 1 | 1 | 1 | |

No of capsules per bottle 30
No of bottles 33

### Example 10

### Preparation of Amyris Alcohol (10%) and Capsaicin Palmitate (0.018%) Oil

The following ingredients were weighed accurately and mixed in a 500 mL beaker while heating at 70° C. The mixture was cooled to room temperature and mixed again to obtain the specified oil.
In a similar manner, oil containing 10% santalyl acetate and 0.018% capsaicin palmitate.

Amyris Alcohol (10%) + Capsaicin
Palmitate (0.018%) Oil Composition

| | | CP Overage (%) 10 | | Bottle (gm) 10 |
|---|---|---|---|---|
| Batch Size (gm) 250.0 | | AA Overage (%) 20 | | Amount |
| Name of Ingredient | | Percent | | Batch (gm) |
| 1 | Cetyl Myristoleate | 10.000 | | 25.000 |
| 2 | Eugenyl Acetate | 1.000 | | 2.500 |
| 3 | Capsaicin Palmitate | 0.018 | 0.020 | 0.050 |
| 4 | Amyris Alcohol | 10.000 | 12.000 | 30.000 |
| 5 | BHT | 0.500 | | 1.250 |
| 6 | Isobornyl Propionate | 1.000 | | 2.500 |
| 5 | Isopropyl Myristate | 10.000 | | 25.000 |
| 6 | Vitamin E Acetate | 2.000 | | 5.000 |
| 7 | Glyceryl Monooleate | 65.482 | 63.480 | 158.701 |
| | TOTAL | 100.000 | | 250.000 |

Total # of Bottle 25

We claim:

1. A pharmaceutical composition comprising:
a) an ester of capsaicin or its analogue or a mixture thereof, and
b) at least one other agent wherein the other agent is selected from the group consisting of a salicylate, menthol, a boswellic acid, DMSO, methyl sulfonylmethane, an NSAID, a corticosteroid, emu oil, an opioid agonist, an opiod antagonist, an NMDA antagonist, tramadol, an α2δ ligand, santalol, santalyl acetate, amyris alcohol, amyris acetate, aloe vera gel and aloe vera juice.

2. The pharmaceutical composition of claim 1 wherein the capsaicin analogue is selected from the group consisting of capsaicin, civamide, homocapsaicin, nordihydrocapsaicin, dihydrocapsaicin, homodihydrocapsaicin, n-vanillyloctanamide, nonivamide and n-vanillyldecanamide.

3. The pharmaceutical composition of claim 1 wherein the ester of capsaicin is selected from the group consisting of capsaicin palmitate, dihydrocapsaicin palmitate and nordihydrocapsaicin palmitate.

4. The pharmaceutical composition of claim 1, wherein the pharmaceutical composition further comprises one or more pharmaceutically acceptable excipients.

5. The pharmaceutical composition of claim 4, wherein the excipients comprise one or more pharmaceutically acceptable antioxidants.

6. The pharmaceutical composition of claim 5, wherein the antioxidant is selected from the group consisting of ascorbic acid, sodium ascorbate, sodium bisulfite, sodium metabisulfate, curcumin, tetrahydrocurcumin, diacetyl tetrahydrocurcumin, resveratrol, quercetin, hesperidin, myricetin, naringin, alpha-lipoic acid and monothioglycerol.

7. The pharmaceutical composition of claim 4, wherein the excipients comprise one or more pharmaceutically acceptable preservatives and/or buffering agents.

8. The pharmaceutical composition of claim 7, wherein the buffering agent is selected from the group consisting of monobasic and dibasic sodium phosphate, sodium benzoate, potassium benzoate, sodium citrate, sodium acetate and sodium tartrate.

9. The pharmaceutical composition of claim 7, wherein the preservative is selected from the group consisting of methylparaben, methylparaben sodium, propylparaben, propylparaben sodium, benzalkonium chloride and benzthonium chloride.

10. The pharmaceutical composition of claim 4, wherein the excipients comprise one or more pharmaceutically acceptable polysaccharides.

11. The pharmaceutical composition of claim 10, wherein the polysaccharide is selected from the group consisting of dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan and sulfated 1,3/1,6-β-D glucan.

12. The pharmaceutical composition of claim 4, wherein the excipients comprise one or more pharmaceutically acceptable skin permeation enhancers.

13. The pharmaceutical composition of claim 12, wherein the skin permeation enhancer is selected from the group consisting of lauryl alcohol, oleyl alcohol, eucalyptol, sodium lauryl sulfate, glyceryl monooleate, sorbitan monooleate, isopropyl myristate, propylene glycol, dimethyl isosorbide and oleic acid.

14. The pharmaceutical composition of claim 1, wherein the pharmaceutical composition is a formulation selected from the group consisting of a topical formulation and an oral formulation.

15. The pharmaceutical composition of claim 14, wherein the oral formulation is selected from the group consisting of a capsule, pill, and elixir formulation.

16. The pharmaceutical composition of claim 1, wherein the amount of ester of capsaicin of part a) is selected from the group consisting of from about 0.1% to about 25% by weight.

17. A method of treating pain associated with post-herpetic neuralgia, diabetic neuropathy, postmastectomy syndrome, oral neuropathiy, trigeminal neuralgia, temperomandibular joint disorders, pruritus, cluster headache, osteoarthritis, arthritis, rhinopathy, oral mucositis, cutaneous allergy, detrusor hyperreflexia, loin pain/hematuria syndrome, neck pain, back pain, amputation stump pain, reflex sympathetic dystrophy and pain due to skin tumor in a subject in need thereof, comprising administering to the subject a therapeutically effective amount of the pharmaceutical composition of claim 1.

18. A method of treating fever blisters, cold sores or herpes in a subject in need thereof, comprising administering to the subject a therapeutically effective amount of the pharmaceutical composition comprising capsaicin palmitate or a mixture of capsaicin palmitate, dihydrocapsaicin palmitate and nordihydrocapsaicin palmitate and at least one other agent selected from the group consisting of santalol, santalyl acetate, amyris alcohol and amyris acetate.

19. The pharmaceutical composition of claim 16, wherein the ester of capsaicin comprises capsaicin palmitate or a mixture of capsaicin palmitate, dihydrocapsaicin palmitate and nordihydrocapsaicin palmitate.
